(19) United States
(12) Patent Application Publication
(10) Pub. No.: US 2025/0205331 A1
(43) Pub. Date: Jun. 26, 2025

(54) NOVEL PLANT VIRUS AND BACTERIOPHAGE VACCINES

(71) Applicant: The Regents of the University of California, Oakland, CA (US)

(72) Inventors: Nicole F. Steinmetz, San Diego, CA (US); Young Hun Chung, San Diego, CA (US)

(21) Appl. No.: 18/852,401

(22) PCT Filed: Mar. 27, 2023
(86) PCT No.: PCT/US2023/016457

§ 371 Date: Sep. 27, 2024

Related U.S. Application Data
(60) Provisional application No. 63/324,580, filed on Mar. 28, 2022.

(51) Int. Cl. A61K 39/385; A61K 39/00; A61K 39/215; A61P 37/04

(52) U.S. Cl. CPC A61K 39/385; A61K 39/0011; A61K 39/215; A61P 37/04; A61K 2039/55; A61K 2039/6075

(57) ABSTRACT

This disclosure provides a conjugate comprising a bacteriophage, plant virus or viral nanoparticle (VNP) conjugated to an antigen or antigenic peptide.

### CROSS-REFERENCE TO RELATED APPLICATIONS

This application claims the benefit under 35 U.S.C. § 119 (e) and under the Paris Convention to U.S. Provisional Application Ser. No. 63/324,580, filed Mar. 28, 2022, the contents of which is incorporated herein by reference in its entirety.

### STATEMENT OF GOVERNMENT SUPPORT

This invention was made with government support under CA224605 and AI161306 awarded by the National Institutes of Health and under DMR2011924 awarded by the National Science Foundation. The government has certain rights in the invention.

### BACKGROUND

The following discussion of the background is merely provided to aid the reader in the understanding the disclosure and is not admitted to describe or constitute prior art to the present disclosure. Throughout and within this disclosure, various patent and technical publications are referenced by an identifying citation or an Arabic number, the full bibliographic citation for which can be found immediately preceding the claims. These disclosures are incorporated herein to more fully describe the state of the art to which this disclosure pertains.

Cowpea mosaic virus (CPMV) is a member of the family Secoviridae. The bipartite positive sense RNA plant virus nanoparticle forms ˜30 nm-sized pseudo-T3 icosahedral proteinaceous shells comprised of 60 copies each of a large (L, 42-kDa) and small (S, 24-kDa) coat protein. Ever since the discovery of CPMV in West Africa in the late 1950s, this nanoscale scaffold has been studied extensively and made impactful progress in many aspects of nanomedicine research. CPMV based nanotechnology has been applied in immunotherapy, vaccines, imaging and drug delivery. Aside from acting as an adjuvant in immunotherapy, CPMV capsids can be engineered to carry active ingredients such as peptides, drugs, fluorophores, and contrast agents. In fact, CPMV was the first plant virus developed as a peptide display system owing to its biocompatibility and high degree of thermal and structural stability.

### SUMMARY OF THE DISCLOSURE

This disclosure provides a conjugate comprising, or consisting essentially of, or consisting of: a virus like particle or VLP (such as a plant virus) or a bacteriophage, the VLP or bacteriophage bound to antigenic peptides through bio-specific interactions using multiple mechanisms such as with nitrilotriacetic acid (NTA)-His tag interactions. Using these bio-specific interactions, a wide array of antigenic peptides against specific diseases, which include, but are not limited to, cancer, cardiovascular diseases, infectious diseases, chronic diseases, autoimmune disorders, and inflammatory disorders can be non-covalently bound to the viral particles.

These novel binding mechanisms improve the development speed and versatility of these viral vaccines allowing for a “plug-and-play” approach where the antigen being bound can be easily swapped out for other antigens depending on the disease and use case. Such versatility has been demonstrated to be extremely important during the COVID-19 pandemic, in which the vaccines that were developed and utilized first, were the ones that demonstrated plug-and-play capabilities. In short, this allows for the use of simple chemistries for the rapid development of a wide variety of viral vaccines that can be tailored to specific diseases.

Most vaccines are still injected as simple mixtures of antigen and adjuvant. However, co-delivery of antigen and adjuvant is vital for improved vaccine efficacy and reduction of negative side effects. This disclosed vaccine approach is able to co-deliver the antigen and adjuvant through bio-specific interactions that are novel and have not been extensively studied in the past. The current approach also is not a chemical conjugation. This is important as it allows us to swap out the desired antigens for a wide variety of other antigens for a “plug-and-play” approach. Chemical conjugations also have to be tailored to the antigen and there can be a multitude of other issues that can delay development. These issues can be, but are not limited to, excessive aggregation of vaccine products, slow development times, inability for a plug-and-play approach, batch-to-batch variability, inaccessibility of antigen epitopes, disruption/breakdown of protein structures, and low antigen conjugation efficiency. The NTA binding has been used with mammalian viruses in the past such as noroviruses. However, in that instance, the virus itself was genetically modified with His-tags, and the drug was conjugated to an NTA molecule. These additional genetic and chemical conjugations may increase the time it takes for development; furthermore, the invention can be used for drug delivery applications, in addition to vaccines. Other instances of using NTA binding for vaccine applications have been studied, but they do not utilize viruses/plant viruses and instead focus on lipid nanoparticles/liposomes. These nanoparticle types may need additional adjuvants if they are non-immunogenic while Applicant's viruses are intrinsically adjuvants.

This disclosure also provides preparations of vaccines for broad applications including but not limited to infectious disease, cancer, cardiovascular disease (CVD), and chronic disease. Simple mixing by use of biospecific interactions allows the binding of antigen and adjuvant for optimal cell targeting to achieve potent efficacy.

Applicant discloses herein a conjugate comprising: 1) a bacteriophage QBeta (“QB”) or virus like particle (VLP) wherein the VLP is or is derived from a plant virus, having one or more external lysines; 2) a histidine-tagged antigen or antigenic peptide; and 3) a nickel nitrilotracetic acid linker (NiNTA linker), wherein the NiNTA linker binds the histidine-tagged antigen or antigenic peptide to one or more external lysines on the bacteriophage QB or VLP. In one embodiment the conjugate comprises a bacteriophage QB. In another embodiment, wherein the conjugate comprises a plant virus that is or is derived from a genus selected from Bromovirus, Comovirus, Tymovirus, or Sobemovirus, optionally wherein the plant virus is or is derived from Cowpea chlorotic mottle virus (CCMV), Cowpea mosaic virus (CPMV), Physalis mottle virus (PhMV), or Sesbania mosaic virus (SeMV). In a further aspect, the plant virus is or is derived from Cowpea mosaic virus (CPMV).

In a further aspect, the conjugate as described herein comprises a viral, bacterial, or tumor antigen or antigenic peptide. Non-limiting examples of such include an antigenic peptide or antigen are selected from a peptide epitope from the SARS-CoV-2 S protein, or a SARS-CoV2-S receptor-binding domain (RBD-domain).

Further provided herein is plurality of the conjugates of as described herein that may be the same or different from each other.

Also provided are compositions comprising, or consisting essentially thereof, or consisting of a conjugate or plurality thereof and a carrier, optionally wherein the carrier is a pharmaceutically acceptable carrier.

The conjugates can be used in a method of inducing an immune response, the method comprising, or consisting essentially thereof, or consisting of administering to a subject in need thereof, an effective amount of one or more of the conjugate, a plurality, a composition as described herein.

The conjugates can be used in a method of treating a subject, the method comprising, or consisting essentially thereof, or consisting of administering to a subject in need thereof, an effective amount of one or more of the conjugate, a plurality, a composition as described herein. In one embodiment, the subject is suffering from cancer and the antigen or antigenic peptide comprise a tumor antigen or antigenic peptide. In another embodiment, the subject is suffering from SARS-CoV-2 and the antigen or antigenic peptide comprises a viral or bacterial antigen, optionally a SARS-CoV-2 S protein, or a SARS-CoV2-S receptor-binding domain (RBD-domain).

Subjects treated by the disclosed methods include mammals such as a human subject.

Further provided is a method of making a vaccine conjugate comprising a bacteriophage QBeta (“QB”) or virus like particle (VLP) having one or more external lysines to a histidine-tagged antigen or antigenic peptide with a nickel nitrilotracetic acid linker (NiNTA linker), wherein the NiNTA linker binds the histidine-tagged antigen or antigenic peptide to one or more external lysines on the bacteriophage QB or VLP. In another aspect, the conjugate is purified or isolated from the reaction mixture and can be combined with a carrier such as a pharmaceutically acceptable carrier.

In one embodiment of the method, the conjugate comprises a bacteriophage QB. In another embodiment, wherein the conjugate comprises a plant virus that is or is derived from a genus selected from Bromovirus, Comovirus, Tymovirus, or Sobemovirus, optionally wherein the plant virus is or is derived from Cowpea chlorotic mottle virus (CCMV), Cowpea mosaic virus (CPMV), Physalis mottle virus (PhMV), or Sesbania mosaic virus (SeMV). In a further aspect, the plant virus is or is derived from Cowpea mosaic virus (CPMV).

In a further aspect, the conjugate of the method as described herein comprises a viral, bacterial, or tumor antigen or antigenic peptide. Non-limiting examples of such include an antigenic peptide or antigen are selected from a peptide epitope from the SARS-CoV-2 S protein, or a SARS-CoV2-S receptor-binding domain (RBD-domain).

### BRIEF DESCRIPTION OF THE FIGURES

FIG. 1: Binding schematic of His-tagged OVA to NiNTA-conjugated CPMV/Qβ. The CPMV, Qβ, and OVA structures were created using Chimera 1.14 (CPMV PDB ID: 1NY7, QB PDB ID: 1QBE, OVA PDB ID: 10VA). The chemical structures were created on ChemDraw 19.0. The small and large CPs of CPMV are shown.

FIGS. 2A-2G: Characterization of NiNTA: His-OVA vaccine formulations. (FIG. 2A) DB of CPMV-NiNTA vs. CPMV against His-OVA on a nitrocellulose membrane. (FIG. 2B) WB against His-tag (left) and OVA (right). (FIG. 2C) SDS-PAGE. In CPMV-NiNTA: His-OVA (left), OVA dissociates from the complex (lane 4); however, in Qβ-NiNTA: His-OVA (right), the CP and OVA remain associated (lane 8). (FIG. 2D) Agarose gel electrophoresis of the vaccine formulations. The increasing molecular weight is better demonstrated by the sloped lines. Left is RNA staining, right is protein staining. (FIG. 2E) Schematic of ELISA. (FIG. 2F) CPMV-NiNTA: His-OVA ELISA and controls. (FIG. 2G) Qβ-NiNTA: His-OVA ELISA and controls. ****=p<0.0001. The schematic in FIG. 2E) was created using Biorender.com.

FIGS. 3A-3C: Antibody titers against OVA. (FIG. 3A) Injection and bleeding schedule. (FIG. 3B) Complete ELISAs at weeks 4 and 6 as well as the endpoint titers. The endpoint titer was determined as the dilution at which the absorbance was greater than twice the blank. The week 2 data can be found in FIG. 14. (FIG. 3C) IgG isotyping. An IgG2b IgG1⁻¹ ratio <1 was considered Th2 while a ratio >1 was considered Th1. The full antibody profile can be found in FIG. 14. *=p<0.05, **=p<0.01, ***=p<0.001, ****=p<0.0001, ns=not significant. The injection schedule schematic was created on Biorender.com.

FIGS. 4A-4C: Tumor volume curves and graphs. (FIG. 4A) Tumor volume curves. (FIG. 4B) Bar graph indicating how long it took before the tumors were discernible for measurement. (FIG. 4C) Bar graph indicating how long it took the tumors to reach a volume of 500 mm³. The CPMV and QB experiments in a-c were all done at the same time, but were separated into two for ease of viewing. The full graphs can be seen in FIG. 16.

FIG. 5: Dithiothreitol (DTT) reduction of CPMV-NiNTA and binding intermediaries. Nickel (Ni), in the presence of DTT, becomes reduced causing a change in color of the solution to brown. While CPMV and CPMV-NTA (no Ni) stay clear, the CPMV-NiNTA solution turns brown indicating that after purification, Ni remains within the solution bound to the CPMV-NTA. Colors not shown.

FIGS. 6A-6B: SDS-PAGE and WB of His-OVA. (FIG. 6A) SDS-PAGE. Following His-tagging of OVA, the molecular weight of the OVA increases slightly resulting in slower electrophoretic mobility. (FIG. 6B) WB against the His-tag. The WB indicates that the His moiety is conjugated to OVA with no background in the native OVA sample.

FIGS. 7A-7B: Demonstration of plug-and-play capabilities of the CPMV/Qβ-NiNTA formulations. (FIG. 7A) SDS-PAGE of Qβ-NiNTA: His-carbonic anhydrase (CA). For Qβ-NiNTA: His-CA, only His-tagged CA is detectable indicating free CA is removed during purification. (FIG. 7B) SDS-PAGE of the Qβ-NiNTA: His-BSA. A characteristic upwards shift of the protein band indicates binding of the CP to BSA (as was observed for OVA, see FIG. 2).

FIGS. 8A-8D: TEM of native and OVA-bound virus particles. TEM of (FIG. 8A) CPMV, (FIG. 8B) CPMV-NiNTA: His-OVA, (FIG. 8C) Qβ, and (FIG. 8D) Qβ-NiNTA: His-OVA. The scale bar in (FIG. 8A) is 100 nm while the scale bars in (FIG. 8A) are at 200 nm.

FIGS. 9A-9C: Circular dichroism spectra of native and OVA-bound virus nanoparticles. Spectra of (FIG. 9A) CPMV-NiNTA: His-OVA, CPMV, and the overlayed graphs, (FIG. 9B) Qβ, Qβ-NiNTA: His-OVA, and the overlayed graphs, and (FIG. 9C) OVA. The overlayed CPMV/QB and the OVA-bound CPMV/Qβ spectra show minimal changes indicating that the binding of OVA does not influence the secondary/tertiary structure of the virus.

FIG. 10: Longitudinal analysis of OVA binding with ELISAs. The absorbance values of the CPMV/Qβ-NiNTA: His-OVA were compared to the absorbance of CPMV/Qβ, respectively, with respect to time. The ELISAs demonstrate that even up to 4 weeks past the generation of the vaccines, they are still able to bind OVA leading to significantly greater signal compared to controls.

FIGS. 11A-11D: Longitudinal analysis of CPMV/Qβ-NiNTA: His-OVA by DLS. (FIG. 11A) Overlayed DLS spectra of CPMV-NiNTA: His-OVA. (FIG. 11B) Overlayed DLS spectra of Qβ-NiNTA: His-OVA. (FIG. 11C) CPMV-NiNTA: His-OVA DLS spectra as a function of time. (FIG. 11D) Qβ-NiTNA: His-OVA DLS spectra as a function of time.

FIGS. 12A-12B: Longitudinal analysis of CPMV/Qβ-NiNTA: His-OVA by FPLC. (FIG. 12A) FPLC graphs of CPMV-NiNTA: His-OVA as a function of time. (FIG. 12B) FPLC graphs of Qβ-NiNTA: His-OVA as a function of time.

FIG. 13: FPLC of native CPMV with unbound OVA. The OVA concentration injected was determined based upon the number of OVA bound per CPMV. At around 20 mL elution volume, there is a small absorbance peak at the 280 nm wavelength indicating the elution of the free OVA.

FIGS. 14A-14B: Week 2 antibody production. (FIG. 14A) ELISA of the week 2 antibody production. (FIG. 14B) Endpoint titers of samples from a). The endpoint titer was determined as the dilution at which the absorbance was twice that of the blank. *=p<0.05, **=p<0.01, ns=not significant. The endpoint titers were analyzed using one-way ANOVA, and all analyses were done on GraphPad Prism.

FIGS. 15A-15E: Full antibody isotyping. Isotyping was accomplished on the antibodies from weeks 2-6 for (FIG. 15A) Qβ-NiNTA: His-OVA, (FIG. 15B) CPMV-NiNTA: His-OVA, (FIG. 15C) Qβ+OVA, (FIG. 15D) CPMV+OVA, and (FIG. 15E) OVA.

FIGS. 16A-16C: Tumor volume curve and delayed onset of tumor development graphs of all groups. (FIG. 16A) Tumor volume curve of all groups. (FIG. 16B) Average number of days until the tumors were palpable. (FIG. 16C) Average number of days until the tumors reached 500 mm³. Tumor volumes were measured using the equation: V=l×w²/2.

FIGS. 17A-17C: Survival curves of the mice in FIG. 4. The survival curves are shown for (FIG. 17A) all groups, (FIG. 17B) only the Qβ groups, and (FIG. 17C) only the CPMV groups. The FIG. 17B, FIG. 17C curves were separated for ease of viewing.

### DETAILED DESCRIPTION

Unless defined otherwise, all technical and scientific terms used herein have the same meanings as commonly understood by one of ordinary skill in the art to which this disclosure belongs. All nucleotide sequences provided herein are presented in the 5′ to 3′ direction. All polypeptide and protein sequences are presented in the direction of the amine terminus to carboxy terminus. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, “and/or” refers to and encompasses any and all possible combinations of one or more of the associated listed items, as well as the lack of combinations when interpreted in the alternative (“or”).

“Optional” or “optionally” means that the subsequently described circumstance may or may not occur, so that the description includes instances where the circumstance occurs and instances where it does not.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

As used herein, the terms “treating,” “treatment” and the like are used herein to mean obtaining a desired pharmacologic and/or physiologic effect. In some embodiments, the effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, e.g., a cancer such as breast cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

RBD stands for the coronavirus spike receptor binding protein.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens. Non-limiting examples of antigens are provided in the below table.

| | Specific Disease or | |
|---|---|---|
| Overarching Disease | Condition | Antigen |
| Autoimmune | Rheumatoid arthritis | TNFα |
| Autoimmune | Rheumatoid arthritis | IL-6 |
| Autoimmune | Rheumatoid arthritis | Multiepitope |
| | | citrullinated |
| | | peptide |
| Autoimmune | Rheumatoid arthritis | Collagen II |
| Autoimmune | Rheumatoid arthritis | RANKL |
| Autoimmune | Rheumatoid arthritis | IL-23 |
| Autoimmune | Rheumatoid arthritis | Human B7-2 |
| | | extracellular |
| | | domain |
| Autoimmune | Rheumatoid arthritis | Proteoglycan epitope |
| Autoimmune | Rheumatoid arthritis | DKK1 |
| Autoimmune | Multiple sclerosis | Myelin |
| Autoimmune | Crohn's disease | TNFα |
| Autoimmune | Crohn's disease | Integrin |
| Autoimmune | Crohn's disease | IL-12 |
| Autoimmune | Crohn's disease | IL-23 |
| Autoimmune | Crohn's disease | IL-17 |
| Autoimmune | Crohn's disease | CCR9 |
| Autoimmune | Crohn's disease | JAK1 |
| Autoimmune | Crohn's disease | JAK3 |
| Autoimmune | Crohn's disease | IL-1β |
| Autoimmune | Crohn's disease | IL-23p19 |
| Autoimmune | Crohn's disease | IFNα |
| Autoimmune | Crohn's disease | VEGF-A |
| Cardiovascular Disease | Atherosclerosis | S100A9 |
| Cardiovascular Disease | Atherosclerosis | LDL |
| Cardiovascular Disease | Atherosclerosis | ApoB-100 |
| Cardiovascular Disease | Atherosclerosis | ApoAl |
| Cardiovascular Disease | Atherosclerosis | Phosphorylcholine |
| | | headgroup |
| Cardiovascular Disease | Atherosclerosis | Cholesteryl |
| | | ester transfer |
| | | protein |
| Cardiovascular Disease | Atherosclerosis | ApoE |
| Cardiovascular Disease | Atherosclerosis | PCSK9 |
| Cardiovascular Disease | Atherosclerosis | Heat shock protein 60 |
| Cardiovascular Disease | Atherosclerosis | Heat shock protein 65 |
| Cardiovascular Disease | Atherosclerosis | T-cell receptor beta |
| | | variable 31 |
| Cardiovascular Disease | Atherosclerosis | CD99 |
| Cardiovascular Disease | Atherosclerosis | VEGF-2 |
| Cardiovascular Disease | Atherosclerosis | Fibronectin |
| Cardiovascular Disease | Stroke | PCSK9 |
| Cardiovascular Disease | Stroke | Angiotensin II |
| Cardiovascular Disease | Stroke | N-methyl-D aspartate |
| Cardiovascular Disease | Stroke | IL-1β |
| Inflammatory Conditions | Fatty liver disease | Oxidized LDL |
| Inflammatory Conditions | Fatty liver disease | CD36 |
| Inflammatory Conditions | Fatty liver disease | SR-A |
| Inflammatory Conditions | Type 2 diabetes mellitus | IL-1β |
| Inflammatory Conditions | Type 2 diabetes mellitus | IL-3 |
| Inflammatory Conditions | Type 2 diabetes mellitus | IL-7 |
| Inflammatory Conditions | Type 2 diabetes mellitus | Dipeptidyl peptidase-4 |
| Inflammatory Conditions | Type 1 diabetes mellitus | B chain of insulin |
| Inflammatory Conditions | Type 1 diabetes mellitus | IA-2 |
| Inflammatory Conditions | Type 1 diabetes mellitus | Glutamic acid |
| | | decarboxylase 65 |
| Inflammatory Conditions | Alzheimer's | Amyloid β |
| Inflammatory Conditions | Alzheimer's | Tau |
| Inflammatory Conditions | Alzheimer's | α-syn |
| Inflammatory Conditions | Parkinson's | α-syn |

The term “tumor or cancer antigen” is an antigen that is expressed on a tumor cell that is expressed at a different level, or not at all on a counterpart normal cell. The term, as used herein, also includes tumor associated antigens. Non-limiting examples of tumor antigens are CD19, mesothelin, ROR1, EGFRVIII, MAGE-D4B, PSMA, HER2, HER3, AFP, CEA CA-125, MUC-1, ETA, MUC-1, BAGE, GAGE-1, MAGE-A1, NY-ESO-1, Gp100, Melan-A/MART-1, Prostate-specific antigen, Mammoglobin-A, Alpha-fetoprotein, HER-2/neu, P53, K-ras, or TRP-2/INT2. Tumor associated antigens (TAAs) are antigens that are present on tumor cells and also normal cells. In some aspects, the TAA may be overexpressed or underexpressed by the tumor cell relative to normal cells. Tumor specific antigens (TSAs) are antigens that may only be expressed by tumor cells and may not be expressed on any other cells. Tumor cell antigens of the instant disclosure include both known and yet to be identified tumor cell antigens. Additional examples are provided in the below table, reproduced in part from Categories of Tumor Antigens, Cancer Medicine, Holland-Frei Cancer Medicine. 6th edition. Kufe D W, Pollock R E, Weichselbaum R R, et al., editors, Copyright 2003, BC Decker Inc.

| Category | Example Antigen | Cancer Histology |
|---|---|---|
| Oncofetal | CEA | Colorectal carcinoma |
| | Immature laminin receptor | RCC |
| | TAG-72 | Prostate carcinoma |
| Oncoviral | HPV E6, E7 | Cervical carcinoma |
| Overexpressed/ | BING-4 | Melanoma |
| accumulated | Calcium-activated | Lung carcinoma |
| | chloride channel 2 | |
| | Cyclin-B₁ | Multi |
| | 9D7 | RCC |
| | Ep-CAM | Breast carcinoma |
| | EphA3 | Multi |
| | Her2/neu | Multi |
| | Telomerase | Multi |
| | Mesothelin | Ductal pancreatic |
| | | carcinoma |
| | SAP-1 | Colorectal carcinoma |
| | Survivin | Multi |
| Cancer-Testis | BAGE family | Multi |
| | CAGE family | Multi |
| | GAGE family | Multi |
| | MAGE family | Multi |
| | SAGE family | Multi |
| | XAGE family | Multi |
| CT9, CT10 | | Multi |
| | NY-ESO-1/LAGE-1 | Multi |
| | PRAME | Multi |
| | SSX-2 | Melanoma, Multi |
| Lineage Restricted | Melan-A/MART-1 | Melanoma |
| | Gp100/pmell7 | Melanoma |
| | Tyrosinase | Melanoma |
| | TRP-1/-2 | Melanoma |
| | P.plypeptide | Melanoma |
| | MC1R | Melanoma |
| | Prostate-pecific antigen | Prostate |
| Mutated | B-catenin | Melanoma, Prostate, HCC |
| | BRCA1/2 | Breast, ovarian carcinoma |
| | CDK4 | Multi |
| | CML66 | CML |
| | Fibronectin | Multi |
| | MART-2 | Melanoma |
| | p53 | Multi |
| | Ras | Multi |
| | TGF-βRII | Colorectal carcinoma |
| Posttranslationally | MUC1 | Ductal carcinoma, RCC |
| altered | | |
| Idiotypic | IG, TCR | B, T leukemia, lymphoma, |
| | | myeloma |

BRCA = breast cancer antigen;
CDK4 = cyclin-dependent kinase-4;
CEA = carcinoembryonic antigen;
CML66 = chronic myelogenous leukemia (antigen) 66;
CT = cancer testis;
HPV = human papilloma virus;
Ep-CAM = epithelial cell adhesion molecule;
Ig = immunoglobulin;
MART-1/-2 = melanoma antigen recognized by T cells-1/-2;
MC1R = melanocortin-1-receptor;
SAP-1 = stomach cancer-associated protein tyrosine phosphatase-1;
TAG-72 = tumor antigen-72;
TCR = T cell receptor;
TGF-βRII = transforming growth factor-β receptor II;
TRP = tyrosinase-related protein.

S100 calcium-binding protein A9 (S100A9; also known as migration inhibitory factor-related protein 14 or MRP14 or calgranulin B) is a protein involved in cellular processes such as cell cycle progression and differentiation and a central mediator of inflammation in cancer and other diseases. It is a calcium-binding protein that regulates inflammation and while there is some level of endogenous S100A9 expression in the squamous epithelium and mucosal tissues, it becomes overexpressed in many different forms of cancer including breast, ovarian, skin, bladder, pancreatic, gastric, esophageal, colon, glioma, cervical, hepatocellular, and thyroid. It is most commonly found in its heterodimer form with S100A8, but can also be found as a homodimer. S100A8/9 complexes are also found in mice and extensive biochemical characterization has demonstrated functional equivalency with its human counterpart. S100A9 expression is heavily linked with tumor aggressiveness and tumorigenesis through the activation of the nuclear factor-κB (NF-κB) and mitogen-activated protein kinase (MAPK) pathways, which are responsible for inflammation-induced cancer development and uncontrolled cell proliferation respectively. It is mainly expressed and secreted by MDSCs, which promotes further accumulation of MDSCs via autocrine pathways into the tumor microenvironment (TME) in an expanding and cyclic fashion. MDSCs suppress the immune response within the TME through reprogramming of the TME into a protumor phenotype, and tumors soon begin establishing S100A9 gradients of myeloid cell migration. In one aspect of this disclosure, the antigen to S100A9 for cancer therapy.

As used herein, the term “antigen binding domain” refers to any protein or polypeptide domain that can specifically bind to an antigen target.

As used herein, the term “autologous,” in reference to cells refers to cells that are isolated and infused back into the same subject (recipient or host). “Allogeneic” refers to non-autologous cells.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

The term “cancer or tumor antigen” refers to an antigen known to be associated and expressed on the surface with a cancer cell or tumor cell or tissue, and the term “cancer or tumor targeting antibody” refers to an antibody that targets such an antigen. Tumor antigens are known in the art and described for example in https://www.cancer.gov/about-cancer/diagnosis-staging/diagnosis/tumor-markers-list, last accessed on Feb. 19, 2023.

Cowpea mosaic virus (CPMV) is a plant-infecting member of the order Picornavirales, with a relatively simple, non-enveloped capsid that has been extensively studied and a positive-sense, single-stranded RNA genome. For CPMV, the genome is bipartite, with RNA-1 (6 kb) and RNA-2 (3.5 kb) being separately encapsidated. CPMV has an icosahedral capsid structure, which is ˜30 nm in diameter and is formed from 60 copies each of a Large (L) and Small(S) coat protein. These two coat proteins are processed from a single RNA-2-encoded precursor polyprotein (VP60) by the action of the 24 K viral proteinase which is encoded by RNA-1. Thus capsid assembly, as well as viral infection, is dependent on the presence of both genomic segments in an infected plant cell.

The terms “CPMV” “CPMV virus” or “CPMV particles” are used interchangeably, referring to a CPMV comprising, or alternatively consisting essentially of, or yet consisting of a capsid and an RNA genome (which is also referred to herein as a viral genome) encapsidated in the capsid. In some embodiments, the CPMV particles have been treated, prepared and/or inactivated by a method as disclosed herein. In some embodiments, the CPMV particle further comprises a heterologous RNA, which is heterologous to (i.e., not naturally presented in) a native CPMV free of any human intervention.

The virus can be obtained according to various methods known to those skilled in the art. In embodiments where plant virus particles are used, the virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393 (2): 329-37 (2009). The disclosures of both of these references are incorporated herein by reference.

As used herein, an anticancer agent refers to any drug or compound used for anticancer treatment. These include any drug that renders or maintains a clinical symptom or diagnostic marker of tumors and cancer, alone or in combination with other compounds, that reduces or maintains a state of remission, reduction, remission, prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin Kl-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S (+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives (as defined for imaging agents) thereof.

As used herein, an ablative therapy is a treatment destroying or ablating cancer tumors. In one embodiment, the ablative therapy does not require invasive surgery. In other embodiments, the ablative therapy refers to removal of a tumor via surgery. In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g. IL-2, interferon's, cytokine inducers).

In some embodiments, a method as disclosed herein further includes the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins.

B16F10 (ATCC® CCL-6475TM) is a muring melanoma cell line from a C57BL/6J mouse. It is a subclone of the B16 tumor line, generated by injecting mice with B16 tumor cells, collecting and culturing secondary tumor growths, and injecting them into fresh mice, a total of 10 times.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

### MODES FOR CARRYING OUT THE DISCLOSURE

Applicant describes herein a conjugate, for example a Qbeta bacteriophage bound to the antigen, non-limiting examples of such include a cancer antigen, a tumor antigen, a viral antigen or ovalbumin, using a NTA linker system that improves significantly titers against the antigen, e.g., the ovalbumin antigen compared to simple mixture of Qbeta and ovalbumin. Another aspect relates to a conjugate comprising another plant virus, e.g., a Cowpea mosaic virus (CPMV).

Methods for making the conjugates are disclosed. For the chemistry, a NTA-polyethylene glycol-N-hydroxysuccinimide (NHS) ester linker was used. The NHS covalently conjugates to the exterior lysines of the virus particle, and the NTA can then be bound to any His-tagged antigen of choice. Due to the prevalence of the His-tag in the biopharmaceutical industry (e.g. for protein purification purposes), this linker can be utilized for a wide array of antigens. In the case of a lack of His-tag, a His-tag can be covalently conjugated directly to the antigen through other chemical procedures.

Antigenic peptides can also be utilized to bind antigens to the bacteriophage or VLP. Applicant has shown that a CPMV binding peptide can be bound to ovalbumin and then subsequently bound to CPMV, and have also demonstrated successful binding with CPMV binding peptides with added COVID epitopes.

The VLPs can be or can be from a plant virus from the group of the genus Bromovirus, Comovirus, or Tymovirus. Non-limiting examples of such include a plant virus selected from Cowpea chlorotic mottle virus (CCMV), Cowpea mosaic virus (CPMV), or Physalis mottle virus (PhMV).

In another aspect, the VLP comprises a capsid protein, optionally a modified capsid protein, further optionally from CCMV, CPMV, PhMV, or a combination thereof.

The antigenic peptide can comprises an extracellular or transmembrane polypeptide that specifically binds to a receptor expressed on a target cell, optionally an immune cell. The immune cell can be an antigen presenting cell, a macrophage, B cell, a dendritic cell, or a nature killer (NK) cell. In one aspect, the macrophage is a tumor-associated macrophage (TAM). The antigen or antigenic peptide can also be a cancer or tumor antigen, e.g.,

In another aspect, the antigenic peptide comprises a peptide that induces an immune response against a pathogen, e.g., when the pathogen is a coronavirus, e.g., SARS-CoV-2.

Provided herein are conjugates comprising, or consisting essentially of, or yet further consisting of, a bacteriophage or a virus-like particle (VLP) derived from a plant virus bound to an antigenic peptide or an antigen. In one aspect, the antigen or antigenic peptide is a histidine tagged antigen or antigenic peptide. Also provided are conjugates comprising a VLP bound to a CBP-functionalized antigen.

Non-limiting examples of antigenic peptides or antigens include a peptide epitope from the SARS-CoV-2 S protein, antigens that target tumor markers or receptors, antigens that target pathogens, ovalbumin or an RBD-domain.

The VLP in the conjugate are from a plant virus from the group of the genus Bromovirus, Comovirus, or Tymovirus. Non-limiting examples of such include a plant virus selected from Cowpea chlorotic mottle virus (CCMV), Cowpea mosaic virus (CPMV), or Physalis mottle virus (PhMV).

In another aspect, the VLP comprises a capsid protein, optionally a modified capsid protein, further optionally from CCMV, CPMV, PhMV, or a combination thereof.

In one aspect, the antigen or antigenic peptide comprises a peptide that induces an immune response against a pathogen, e.g., when the pathogen is a coronavirus, e.g., SARS-CoV-2. In another aspect, the antigenic peptide comprises, or consists essentially of, or yet further consists of, a B-cell epitope selected from amino acids 553-570, 625-636 or 809-826.

More than one antigenic peptide or antigen can be bound to the VLP or bacteriophage, that may be the same or different from each other and can be selected to treat the same or different medical condition.

Also provided are compositions comprising one or more bacteriophage and/or VLP conjugates as described herein, and a carrier, optionally a pharmaceutically acceptable carrier. In one aspect, the composition is formulated for in vitro or in vivo use, optionally systemic administration. In one embodiment, the composition is formulated for local administration. In another embodiment, the composition is formulated for parenteral administration, optionally for intravenous, subcutaneous, intramuscular, intracerebral, intranasal, intra-arterial, intra-articular, intradermal, intravitreal, intraosseous infusion, intraperitoneal, or intrathecal administration.

In one aspect, the composition further comprises a preservative or stabilizer, that can be lyophilized or frozen.

The compositions can be used for treating a disease or condition or inducing an immune response in a subject in need thereof, comprising, or consisting essentially of, or yet further consisting of, administering to the subject a formulation or composition as described above and herein. Non-limiting examples of such include cancer (e.g., metastatic or primary, e.g., colon cancer), an inflammatory condition, an autoimmune disease, an allergy, or a pathogenic infection. In one aspect, the therapeutic peptide induces an immune response to induce an immune response for the treating or preventing a COVID infection. In another aspect, the formulation does not comprise a therapeutic peptide and the disease is cancer, e.g., colon cancer. The compositions can be combined with other appropriate therapies, in the same or different composition or formulation.

The method further comprises preparing compositions by admixes one or more formulations as described herein, with a carrier, optionally a pharmaceutically acceptable carrier.

In one aspect, the method further admixing a preservative or stabilizer. The method further comprises lyophilizing or freezing the formulation or composition.

Further provide are kits comprising the formulations or the compositions as described herein, and instructions for use.

### Methods of Making the Vaccine Conjugate

Provided herein is a method of making a vaccine conjugate, the method comprising, or consisting essentially of, or yet further consisting of conjugating a bacteriophage QBeta (“Qβ”) or virus nanoparticle (VNP) having one or more external lysines to a histidine-tagged antigen or antigenic peptide with a nickel nitrilotracetic acid linker (NiNTA linker), wherein the NiNTA linker binds the histidine-tagged antigen or antigenic peptide to one or more external lysines on the bacteriophage Qβ or VNP. In one aspect, the conjugate is or comprises a bacteriophage Qβ. In another aspect, the conjugate is or comprises CPMV. In a further aspect, the VNP is or is derived from a plant virus. Non-liming examples of plant virus comprises or is derived from a genus selected from Bromovirus, Comovirus, Tymovirus, or Sobemovirus, optionally wherein the plant virus is or is derived from Cowpea chlorotic mottle virus (CCMV), Cowpea mosaic virus (CPMV), Physalis mottle virus (PhMV), or Sesbania mosaic virus (SeMV). In a particular aspect, the plant virus is or is derived from Cowpea mosaic virus (CPMV).

With respect to antigens, the antigen or antigenic peptide comprises a viral, bacterial, or tumor antigen or antigenic peptide. Non-limiting examples of such are disclosed above and include a peptide epitope from the SARS-CoV-2 S protein, or a SARS-CoV2-S receptor-binding domain (RBD-domain), a tumor antigen, an antigen to treat infectious disease, an antigen to treat cardiovascular disease, or an antigen to treat inflammation. The antigens for use in the method can be the same or different from each other and selected to treat the same or different diseases or conditions.

Further provided is one or more conjugates prepared by the methods disclosed herein.

Applicant has found that the disclosed method provides a quick and effective means to provide therapeutic vaccine molecules that provide therapeutic benefit when administered to patients in need thereof. The efficiency and simplicity of the method allows for the generation of vaccine panels for testing for personalized therapies. The binding is not a conjugation, which means that time-consuming and non-adaptable methods are not required to bind the antigen to the adjuvant. This will improve the speed of manufacture of the vaccine, which as seen by the pandemic was quite important. Also, conjugation can lead to batch-to-batch variability, epitope masking, aggregation, and disruption of protein structures. An alternative to conjugation is to use peptide epitopes, but these vaccines usually have a narrowed breadth of response and limited neutralization. The current chemistry still allows for antibody production against the entire protein given that the protein is being bound using the NTA: His-tag chemistry. The present disclosure also provide for co-delivery of the antigen and adjuvant to the same cell, which boosts antibody response. This binding procedure allows for plug-and-play of the antigen. In theory, any antigen as long as it has a histidine tag should be able to be bound to the CPMV or Qbeta without changing the chemistry. This also improves the speed of the vaccine manufacturing process as the same chemistry can be used for any antigen of interest, see for example, the binding of the BSA and carbonic anhydrase. These disclosed vaccine conjugates, e.g., CPMV and Qbeta, are potent adjuvants that have demonstrated long-lasting, durable antibody responses.

### Virus-Like Particles (VLPs)

As utilized herein, a VLP is a non-native VLP that comprise, or consists essentially of, or yet further consists of, one or more viral particles, e.g., a capsid, derived from a plant virus. In some instances, the plant virus is from the genus Bromovirus, Comovirus, Tymovirus, or Sobemovirus. In some cases, the VLP is or is derived from Cowpea chlorotic mottle virus (CCMV), Cowpea mosaic virus (CPMV), Physalis mottle virus (PhMV), or Sesbania mosaic virus (SeMV).

In some instances, the VLP comprise, or consists essentially of, or yet further consists of, a capsid protein derived from a plant virus. In some instances, the capsid protein is a wild-type protein derived from the plant virus. In other instances, the capsid protein is a variant of the wild-type protein derived from the plant virus. In additional instances, the capsid protein is a modified protein, either full-length or truncated version.

As used herein, the term “Virus-like particle” or “VLP” refers to a non-replicating, viral shell, derived from one or more viruses (e.g., one or more plant viruses described herein). VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. VLPs can also be engineered, e.g., comprising, or consisting essentially of, or yet further consisting of, one or more viral proteins that comprise, or consists essentially of, or yet further consists of, a modification. Methods for producing VLPs are known in the art. The presence of VLPs following recombinant expression of viral proteins can be detected using conventional techniques known in the art, such as by electron microscopy, biophysical characterization, and the like. Further, VLPs can be isolated by known techniques, e.g., density gradient centrifugation and identified by characteristic density banding. See, for example, Baker et al. (1991) Biophys. J. 60:1445-1456; and Hagensee et al. (1994) J. Viral. 68:4503-4505; Vincente, J Invertebr Pathol., 2011; Schneider Ohrum and Ross, Curr. Top. Microbial. Immunol., 354:53073, 2012).

In some embodiments, the VLP is derived from Cowpea chlorotic mottle virus (CCMV). CCMV is a spherical plant virus that belongs to the Bromovirus genus. Several strains have been identified and include, but not limited to, Carl (Ali, et al., 2007. J. Virological Methods 141:84-86), Car2 (Ali, et al., 2007. J. Virological Methods 141:84-86, 2007), type T (Kuhn, 1964. Phytopathology 54:1441-1442), soybean(S) (Kuhn, 1968. Phytopathology 58:1441-1442), mild (M) (Kuhn, 1979. Phytopathology 69:621-624), Arkansas (A) (Fulton, et al., 1975. Phytopathology 65:741-742), bean yellow stipple (BYS) (Fulton, et al., 1975. Phytopathology 65:741-742), R (Sinclair, ed. 1982. Compendium of Soybean Diseases. 2^{nd} ed. The American Phytopathological Society, St. Paul. 104 pp.), and PSM (Paguio, et al., 1988. Plant Diseases 72 (9): 768-770).

In some instances, the VLP from CCMV comprise, or consists essentially of, or yet further consists of, a plurality of capsid proteins. In some instances, the capsid protein is a wild-type CCMV capsid, optionally expressed by Car1, Car2, type T, soybean(S), mild (M), Arkansas (A), bean yellow stipple (BYS), R, or PSM strain. In other instances, the capsid protein is a modified capsid protein, e.g., comprising, or consisting essentially of, or yet further consisting of, one or more substitutions, insertions, and/or deletions. In some cases, the CCMV capsid comprise, or consists essentially of, or yet further consists of, s the sequence as set forth in the UniProtKB ID P03601:

(SEQ ID NO: )
MSTVGTGKLTRAQRRAAARKNKRNTRVVQPVIVEPIASGQGKAIK
AWTGYSVSKWTASCAAAEAKVTSAITISLPNELSSERNKQLKVGR
VLLWLGLLPSVSGTVKSCVTETQTTAAASFQVALAVADNSKDVVA
AMYPEAFKGITLEQLTADLTIYLYSSAALTEGDVIVHLEVEHVRP
TFDDSFTPVY,

or an equivalent thereof.

In some cases, the VLP from CCMV is prepared by the method as described in Ali et al., “Rapid and efficient purification of Cowpea chlorotic mottle virus by sucrose cushion ultracentrifugation,” Journal of Virological Methods 141:84-86 (2007).

In some embodiments, the VLP is or is derived from Cowpea mosaic virus (CPMV). CPMV is a non-enveloped plant virus that belongs to the Comovirus genus. CPMV strains include, but are not limited to, SB (Agrawal, H. O. (1964). Meded. Landb. Hoogesch. Wagen. 64:1) and Vu (Agrawal, H. O. (1964). Meded. Landb. Hoogesch. Wagen. 64:1).

In some instances, the VLP from CPMV comprise, or consists essentially of, or yet further consists of, a plurality of capsid proteins. In some instances, CPMV produces a large capsid protein and a small capsid protein precursor (which generates a mature small capsid protein). In some cases, CPMV capsid is formed from a plurality of large capsid proteins and mature small capsid proteins. In some cases, the large capsid protein is a wild-type large capsid protein, optionally expressed by SB or Vu strain. In other instances, the large capsid protein is a modified large capsid protein, e.g., comprising, or consisting essentially of, or yet further consisting of, one or more substitutions, insertions, and/or deletions. In some cases, the large capsid protein comprise, or consists essentially of, or yet further consists of, the sequence as set forth in the UniProtKB ID P03599 (residues 460-833):

(SEQ ID NO: )
MEQNLFALSLDDTSSVRGSLLDTKFAQTRVLLSKAMAGGDVLLDE
YLYDVVNGQDFRATVAFLRTHVITGKIKVTATTNISDNSGCCLML
AINSGVRGKYSTDVYTICSQDSMTWNPGCKKNFSFTFNPNPCGDS
WSAEMISRSRVRMTVICVSGWTLSPTTDVIAKLDWSIVNEKCEPT
IYHLADCQNWLPLNRWMGKLTFPQGVTSEVRRMPLSIGGGAGATQ
AFLANMPNSWISMWRYFRGELHFEVTKMSSPYIKATVTFLIAFGN
LSDAFGFYESFPHRIVQFAEVEEKCTLVFSQQEFVTAWSTQVNPR
TTLEADGCPYLYAIIHDSTTGTISGDFNLGVKLVGIKDFCGIGSN
PGIDGSRLLGAIAQ,

or an equivalent thereof.

In some cases, the mature small capsid protein is a wild-type mature small capsid protein, optionally expressed by SB or Vu strain. In other instances, the mature small capsid protein is a modified mature small capsid protein, e.g., comprising, or consisting essentially of, or yet further consisting of, one or more substitutions, insertions, and/or deletions. In some cases, the mature small capsid protein comprise, or consists essentially of, or yet further consists of, s the sequence as set forth in the UniProtKB ID P03599 (residues 834-1022):

(SEQ ID NO: )
GPVCAEASDVYSPCMIASTPPAPFSDVTAVTFDLINGKITPVGDD
NWNTHIYNPPIMNVLRTAAWKSGTIHVQLNVRGAGVKRADWDGQV
FVYLRQSMNPESYDARTFVISQPGSAMLNFSFDIIGPNSGFEFAE
SPWANQTTWYLECVATNPRQIQQFEVNMRFDPNFRVAGNILMPPF
PLSTETPPL,

or an equivalent thereof.

In some embodiments, the VLP is derived from Physalis mottle virus (PhMV). PhMV is a single stranded RNA virus that belongs to the genus Tymovirus. In some instances, the VLP from PhMV comprises, or consists essentially of, or yet further consists of, a plurality of coat proteins. In some instances, the coat protein is a wild-type PhMV coat protein. In other instances, the coat protein is a modified coat protein, e.g., comprising, or consisting essentially of, or yet further consisting of, one or more substitutions, insertions, and/or deletions. In some cases, the PhMV coat comprise, or consists essentially of, or yet further consists of, s the sequence as set forth in the UniProtKB ID P36351:

(SEQ ID NO: )
MDSSEVVKVKQASIPAPGSILSQPNTEQSPAIVLPFQFEATTFGT
AETAAQVSLQTADPITKLTAPYRHAQIVECKAILTPTDLAVSNPL
TVYLAWVPANSPATPTQILRVYGGQSFVLGGAISAAKTIEVPLNL
DSVNRMLKDSVTYTDTPKLLAYSRAPTNPSKIPTASIQISGRIRL
SKPMLIAN,

or an equivalent thereof.

In some embodiments, the VLP is derived from Sesbania mosaic virus (SeMV). SeMV is a positive stranded RNA virus that belongs to the genus Sobemovirus. In some instances, the VLP from SeMV comprise, or consists essentially of, or yet further consists of, a plurality of capsid proteins. In some instances, the capsid protein is a wild-type SeMV capsid protein. In other instances, the capsid protein is a modified capsid protein, e.g., comprising, or consisting essentially of, or yet further consisting of, one or more substitutions, insertions, and/or deletions. In some cases, the SeMV capsid comprise, or consists essentially of, or yet further consists of, the sequence as set forth in the UniProtKB ID Q9EB06:

(SEQ ID NO: )
MAKRLSKQQLAKAIANTLETPPQPKAGRRRNRRRQRSAVQQLQPT
QAGISMAPSAQGAMVRIRNPAVSSSRGGITVLTHSELSAEIGVTD
SIVVSSELVMPYTVGTWLRGVAANWSKYSWLSVRYTYIPSCPSST
AGSIHMGFQYDMADTVPVSVNQLSNLRGYVSGQVWSGSAGLCFIN
GTRCSDTSTAISTTLDVSKLGKKWYPYKTSADYATAVGVDVNIAT
PLVPARLVIALLDGSSSTAVAAGRIYCTYTIQMIEPTASALNN,

or an equivalent thereof.

As used herein, the term “an equivalent thereof” in reference to a polynucleotide or a protein (e.g., a capsid or coat protein) include a polynucleotide or a protein that comprise, or consists essentially of, or yet further consists of, at least 80%, 85%, 90%, 95%, 96%, 97%, 98%, or 99% sequence identify to the respective polynucleotide or protein of which it is compared to, while still retaining a functional activity. In the instances with reference to a capsid or coat protein, a functional activity refers to the formation of a VLP.

As used herein, the term “modification” include, for example, substitutions, additions, insertions and deletions to the amino acid sequences, which can be referred to as “variants.” Exemplary sequence substitutions, additions, and insertions include a full length or a portion of a sequence with one or more amino acids substituted (or mutated), added, or inserted, for example of a capsid derived from the plant virus. In some instances, a capsid described herein includes, e.g., a modified capsid comprising, or consisting essentially of, or yet further consisting of, at least 70%, 80%, 85%, 90%, 95%, 96%, 97%, 98%, 99%, or 100% sequence identity to its respective wild-type version.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/blast/Blast.cgi.

Modified capsid polypeptides include, for example, non-conservative and conservative substitutions of the capsid amino acid sequences.

As used herein, the term “conservative substitution” denotes the replacement of an amino acid residue by another, chemically or biologically similar residue. Biologically similar means that the substitution does not destroy a biological activity or function, e.g., assembly of a viral capsid.

Structurally similar means that the amino acids have side chains with similar length, such as alanine, glycine and serine, or a similar size. Chemical similarity means that the residues have the same charge or are both hydrophilic or hydrophobic. Particular examples of conservative substitutions include the substitution of a hydrophobic residue such as isoleucine, valine, leucine or methionine for another, the substitution of a polar residue for another, such as the substitution of arginine for lysine, glutamic for aspartic acids, or glutamine for asparagine, and the like. The term “conservative substitution” also includes the use of a substituted amino acid in place of an unsubstituted parent amino acid. Such proteins that include amino acid substitutions can be encoded by a nucleic acid. Consequently, nucleic acid sequences encoding proteins that include amino acid substitutions are also provided.

Modified proteins also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Modified forms further include “chemical derivatives,” in which one or more amino acids has a side chain chemically altered or derivatized. Such derivatized polypeptides include, for example, amino acids in which free amino groups form amine hydrochlorides, p-toluene sulfonyl groups, carobenzoxy groups; the free carboxy groups form salts, methyl and ethyl esters; free hydroxl groups that form O-acyl or O-alkyl derivatives as well as naturally occurring amino acid derivatives, for example, 4-hydroxyproline, for proline, 5-hydroxylysine for lysine, homoserine for serine, ornithine for lysine etc. Also included are amino acid derivatives that can alter covalent bonding, for example, the disulfide linkage that forms between two cysteine residues that produces a cyclized polypeptide.

In some instances, a VLP described herein further comprise, or consists essentially of, or yet further consists of, a label or a tag, e.g., such as a detectable label. A detectable label can be attached to, e.g., to the surface of a VLP.

Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Fe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

As set forth herein, a detectable label or tag can be linked or conjugated (e.g., covalently) to the VLP. In various embodiments a detectable label, such as a radionuclide or metal or metal oxide can be bound or conjugated to the agent, either directly or indirectly. A linker or an intermediary functional group can be used to link the molecule to a detectable label or tag. Linkers include amino acid or peptidomimetic sequences inserted between the molecule and a label or tag so that the two entities maintain, at least in part, a distinct function or activity. Linkers may have one or more properties that include a flexible conformation, an inability to form an ordered secondary structure or a hydrophobic or charged character which could promote or interact with either domain. Amino acids typically found in flexible protein regions include Gly, Asn and Ser. The length of the linker sequence may vary without significantly affecting a function or activity.

Linkers further include chemical moieties, conjugating agents, and intermediary functional groups. Examples include moieties that react with free or semi-free amines, oxygen, sulfur, hydroxy or carboxy groups. Such functional groups therefore include mono and bifunctional crosslinkers, such as sulfo-succinimidyl derivatives (sulfo-SMCC, sulfo-SMPB), in particular, disuccinimidyl suberate (DSS), BS3 (Sulfo-DSS), disuccinimidyl glutarate (DSG) and disuccinimidyl tartrate (DST). Non-limiting examples include diethylenetriaminepentaacetic acid (DTPA) and ethylene diaminetetracetic acid.

Also provided herein is the VLP or bacteriophage as described herein in a composition that further comprises, or consists essentially of, or yet further consists of an additional therapeutic agent.

In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

In some cases, the VLP or bacteriophage with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, chemotherapy. In other cases, the first-line treatment comprise, or consists essentially of, or yet further consists of, radiation therapy. A skilled artisan would readily understand that different first-line treatments may be applicable to different type of cancers.

In some cases, the additional therapeutic agent comprise, or consists essentially of, or yet further consists of, or is used as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In connection with cancer care, the treatment can comprise an additional therapeutic agent that comprises, or consists essentially of, or yet further consists of, an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULAR, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, s an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM.

In some cases, the additional therapeutic agent comprise, or consists essentially of, or yet further consists of, s pembrolizumab, nivolumab, tremelimumab, or ipilimumab.

In some cases, the additional therapeutic agent comprise, or consists essentially of, or yet further consists of, s an antibody such as alemtuzumab, trastuzumab, ibritumomab tiuxetan, brentuximab vedotin, ado-trastuzumab emtansine, or blinatumomab.

In some cases, the additional therapeutic agent comprise, or consists essentially of, or yet further consists of, a cytokine. Exemplary cytokines include, but are not limited to, IL-Iβ, IL-6, IL-7, IL-10, IL-12, IL-15, IL-21, or TNFα.

In some embodiments, the additional therapeutic agent comprise, or consists essentially of, or yet further consists of, a receptor agonist. In some instances, the receptor agonist comprise, or consists essentially of, or yet further consists of, a Toll-like receptor (TLR) ligand. In some cases, the TLR ligand comprise, or consists essentially of, or yet further consists of, s TLR1, TLR2, TLR3, TLR4, TLR5, TLR6, TLR7, TLR8, or TLR9. In some cases, the TLR ligand comprise, or consists essentially of, or yet further consists of, s a synthetic ligand such as, for example, Pam3Cys, CFA, MALP2, Pam2Cys, FSL-1, Hib-OMPC, Poly I: C, poly A: U, AGP, MPL A, RC-529, MDF2p, CFA, or Flagellin.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the additional therapeutic agent is, or can be used as a vaccine, optionally, an oncolytic virus. Exemplary oncolytic viruses include T-Vec (Amgen), G47A (Todo et al.), JX-594 (Sillajen), CG0070 (Cold Genesys), and Reolysin (Oncolytics Biotech).

In some instances, the VLP or bacteriophage composition or formulation as described herein is administered in combination with a radiation therapy.

In some instances, the VLP formulation described herein is administered in combination with surgery.

In some instances, an additional therapeutic agent in the context of a pathogenic infection comprise, or consists essentially of, or yet further consists of, an antibiotics or an antiviral treatments such as, but not limited to, acyclovir, brivudine, docosanol, famciclovir, foscarnet, idoxuridine, penciclovir, trifluridine, valacyclovir, and pritelivir.

In some instances, the pathogen is human immunodeficiency virus (HIV). In some cases, the additional therapeutic agent comprise, or consists essentially of, or yet further consists of, s an HIV antiretroviral therapy. Exemplary HIV antiretroviral therapy includes: nucleoside reverse transcriptase inhibitors (RTIs) such as abacavir, emtricitabine, lamivudine, tenofovir disoproxil fumarate, and zidovudine; non-nucleoside reverse transcriptase inhibitors (NNRTIs) such as efavirenz, etravirine, nevirapine, or rilpivirine; protease inhibitors (Pis) such as atazanavir, darunavir, fosamprenavir, ritonavir, saquinavir, and tipranavir; fusion inhibitors such as enfuvirtide; CCR5 antagonists such as maraviroc; integrase inhibitors such as dolutegravir and raltegravir; post-attachment inhibitors such as ibalizumab; pharmacokinetic enhancers such as cobicistat; and cocktails such as abacavir and lamivudine; abacavir, dolutegravir, and lamivudine; abacavir, lamivudine, and zidovudine; atazanavir and cobicistat; bictegravir, emtricitabine, and tenofovir alafenamide; darunavir and cobicistat; dolutegravir and rilpivirine; efavirenz, emtricitabine, and tenofovir disoproxil fumarate; efavirenz, lamivudine, and tenofovir disoproxil fumarate; efavirenz, lamivudine, and tenofovir disoproxil fumarate; elvitegravir, cobicistat, emtricitabine, and tenofovir alafenamide fumarate; elvitegravir, cobicistat, emtricitabine, and tenofovir disoproxil fumarate; emtricitabine, rilpivirine, and tenofovir alafenamide; emtricitabine, rilpivirine, and tenofovir disoproxil fumarate; emtricitabine and tenofovir alafenamide; emtricitabine and tenofovir disoproxil fumarate; lamivudine and tenofovir disoproxil fumarate; lamivudine and zidovudine; and lopinavir and ritonavir.

In some instances, the pathogen is a hepatitis virus, e.g., hepatitis A, B, C, D, or E. In some cases, an additional therapeutic agent comprise, or consists essentially of, or yet further consists of, an antiviral therapy for hepatitis. Exemplary antiviral therapy for hepatitis include ribavirin; NS3/4A protease inhibitors such as paritaprevir, simeprevir, and grazoprevir; NS5A protease inhibitors such as ledipasvir, ombitasvir, elbasvir, and daclatasvir; NS5B nucleotide/nucleoside and nonnucleoside polymerase inhibitors such as sofosbuvir and dasabuvir; and combinations such as ledipasvir-sofosbuvir, dasabuvir-ombitasvir-paritaprevir-ritonavir; elbasvir-grazoprevir, ombitasvir-paritaprevir-ritonavir, sofosbuvir-velpatasvir, sofosbuvir-velpatasvir-voxilaprevir, and glecaprevir-pibrentasvir; and interferons such as peginterferon alfa-2a, peginterferon alfa-2b, and interferon alfa-2b.

In one aspect, the pathogen is a coronavirus, e.g., COVID-2.

Exemplary inflammatory conditions include autoimmune disease or disorder include, but are not limited to those identified herein (and incorporated herein by reference) and include, alopecia areata, autoimmune hemolytic anemia, autoimmune hepatitis, dermatomyositis, type 1 diabetes, juvenile idiopathic arthritis, glomerulonephritis, Graves' disease, Guillain-Barre syndrome, idiopathic thrombocytepenic purpura, myasthenia gravis, multiple sclerosis, pemphigus/pemphigoid, pernicious anemia, polyarteritis nodosa, polymyositis, primary biliary cirrhosis, psoriasis, rheumatoid arthritis, scleroderma, Sjogren's syndrome, systemic lupus erythematosus, thyroiditis, uveitis, vitiligo, or Wegener's granulomatosis.

Exemplary additional therapeutic agents for the treatment of an autoimmune disease or disorder include, but are not limited to, corticosteroids such as prednisone, budesonide, or prednisolone; calcineurin inhibitors such as cyclosporine or tacrolimus; mTOR inhibitors such as sirolimus or everolimus; EVIDH inhibitors such as azathioprine, leflunomide, or mycophenolate; biologies such as abatacept, adalimumab, anakinra, certolizumab, etanercept, golimumab, infliximab, ixekizumab, natalizumab, rituximab, secukinumab, tocilizumab, ustekinumab, or vedolizumab; and monoclonal antibodies such as basiliximab, daclizumab, or muromonab.

Exemplary inflammatory conditions include, but are not limited to, asthma, chronic peptid ulcer, tuberculosis, rheumatoid arthritis, ulcerative colitis, and Crohn's disease.

### Compositions

Also provided herein are the compositions containing the conjugates comprising, or consisting essentially of, or yet further consisting of, the antigenic peptide or antigen bound to the VLP or bacteriophage as described herein alone or in combination with the additional therapeutic agents. In one aspect, the conjugates further comprise, or consist essentially of, or yet further consist of, a carrier, such as a pharmaceutically acceptable carrier.

In another aspect, provided herein is a composition comprising, consisting essentially of, or consisting of the antigenic peptide or antigen bound to the VLP or bacteriophage as described herein alone or in combination and at least one pharmaceutically acceptable excipient or pharmaceutically acceptable carrier.

In another embodiment, this technology relates to a pharmaceutical composition comprising an effective amount or a therapeutically effective amount of a combination of the antigenic peptide or antigen bound to the VLP or bacteriophage as described herein alone or in combination and a pharmaceutically acceptable carrier.

Compositions, including pharmaceutical compositions comprising, consisting essentially of, or consisting of the VLP formulation alone or in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical compositions. described herein are administered to a subject by multiple administration routes, including but not limited to, parenteral, oral, buccal, rectal, sublingual, or transdermal administration routes. In some cases, parenteral administration comprise, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington ‘s Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL®PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorb ate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the conjugates or compositions may be administered to a subject suffering from a condition as disclosed herein, such as a human, either alone or as part of a pharmaceutically acceptable formulation, once a week, once a day, twice a day, three times a day, or four times a day, or even more frequently.

Administration of the conjugates alone or in combination with the additional therapeutic agent and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprise, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis.

The conjugates or compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

### Uses of the Conjugates and Compositions Containing Same

Provided herein is a method of treating a disease or condition or inducing an immune response in a cell or a subject in need thereof, comprising, or consisting essentially of, or yet further consisting of administering to the subject or cell (as appropriate) a conjugate comprising a VLP and/or bacteriophage as described herein joined to an antigenic peptide or antigen selected to induce the immune response, or a composition as described herein.

In one aspect, the cell or disease or condition is a cancer cell, or a cancer or tumor, e.g. a solid tumor and the antigen or antigenic peptide in the conjugate is a cancer or tumor antigen selected to treat the cancer or tumor. Non-limiting examples of a solid tumors or cells are bladder cancer, bone cancer, brain cancer, breast cancer, colorectal cancer, colon cancer, esophageal cancer, eye cancer, head and neck cancer, kidney cancer, lung cancer, melanoma, ovarian cancer, pancreatic cancer, prostate cancer, or stomach cancer. In a further aspect, the solid tumor is a colon cancer, pancreatic cancer or melanoma. In a further aspect, the cancer or cell is a hematologic malignancy, such as, for example, a lymphoma or leukemia. Non-limiting examples include a B-cell lymphoma, a T-cell lymphoma, a Hodgkin's lymphoma or a non-Hodgkin's lymphoma. The cancer can be primary or metastatic, e.g., Stage I, Stage II, Stage III or Stage IV. It also can be relapsed or refractory cancer.

The compositions can be used to test therapies in vitro as described herein. The cell in the assay can be a primary cell obtained from example, a biopsy or an established cell line obtained from example a commercial source such as the American Type Culture Collection (ATCC).

In one aspect, the method or conjugate composition or formulation modulates, impedes, or inhibits the growth of a cancer cell or tumor growth and the antigen or antigenic peptide induces a response to the tumor or cancer. In a yet further aspect, the VLP formulation promotes accumulation of tumor-infiltrating lymphocytes in the tumor microenvironment. In one aspect, the method is practiced in vitro and can serve as an assay to test new combination therapies or dosage regimens. It also can be used as a personalized assay by administering to a subject's cancer or tumor cell in vitro the VLP or bacteriophage joined to the antigen or antigenic peptide, or composition containing same to the cell. One of skill in the art can use conventional assays, such as reduction in tumor size, tumor burden or a reduction in an appropriate cancer marker to determine if the method is appropriate for the subject in need of such treatment. The methods can be practiced clinically, e.g. in a human subject or in laboratory animals as an animal model to test for new combination therapies. One of skill in the art can use conventional assays, such as reduction in tumor size, tumor burden or a reduction in an appropriate cancer marker to determine if the method is appropriate for the subject in need of such treatment or if the treatment has been successful or requires repeating or a change in dosage.

In a further aspect, the conjugate comprising the bacteriophage and/or VLP joined to the antiviral antigen or antigenic peptide, or composition containing same, induces an immune response in the subject in need thereof, e.g., an immune response against a coronavirus, e.g., a COVID-2 infection and the antigen or antigenic peptide raises an immune response to the COVID-2 infection. In one aspect, the antigenic peptide is a fragment of the SARs S protein.

The methods can be practiced clinically in a human subject for example or in laboratory animals as an animal model to test for new combination therapies. One of skill in the art can use conventional assays for measuring immune responses.

In the context of cancer care, the subject of these methods can be an animal, a mammal or a human in need of such treatment. When the methods are practiced in vitro, the cell can be an animal cell, a mammalian cell or a human cell. In one aspect an effective amount is administered which can be determined using conventional techniques. When the treatment relates to cancer therapy, the method or treatment can be a first-line, second-line, third-line or fourth-line therapy. In can be an adjuvant therapy and combined with other therapies as determined by the treating veterinarian or physician. Any appropriate means of administration can be used, also as determined by the treating veterinarian or physician. The treatments can be combined with diagnostic assessment before or after therapy. Thus, the therapy can be personalized to the subject in need of such treatment.

Provided herein is a method of treating an inflammatory condition or infectious disease in a subject in need thereof, comprising, or consisting essentially of, or yet further consisting of administering to the subject an antiviral or other antigen or antigenic peptide to raise an immune response to the virus or infectious agent joined to the bacteriophage and/or VLP or a composition as described herein. The methods can be practiced clinically, e.g. in a human subject or in laboratory animals as an animal model to test for new combination therapies. Methods to measure inflammatory responses are known in the art. In the context of autoimmune or infectious disease, the subject of these methods can be an animal, a mammal or a human in need of such treatment. When the methods are practiced in vitro, the cell can be an animal cell, a mammalian cell or a human cell. In one aspect an effective amount is administered which can be determined using conventional techniques. The therapies can be combined with other therapies as determined by the treating veterinarian or physician. Any appropriate means of administration can be used, also as determined by the treating veterinarian or physician. The treatments can be combined with diagnostic assessment before or after therapy. Thus, the therapy can be personalized to the subject in need of such treatment.

Further provided is a method of treating an infectious disease in a subject in need thereof, comprising, or consisting essentially of, or yet further consisting of administering to the subject an antigen or antigenic peptide selected to treat the infectious disease joined to the bacteriophage and/or VLP or a composition as described herein. The methods can be practiced clinically, e.g. in a human subject or in laboratory animals as an animal model to test for new combination therapies. Methods to measure inflammatory responses are known in the art. In the context of autoimmune or infectious disease, the subject of these methods can be an animal, a mammal or a human in need of such treatment. When the methods are practiced in vitro, the cell can be an animal cell, a mammalian cell or a human cell. In one aspect an effective amount is administered which can be determined using conventional techniques. The therapies can be combined with other therapies as determined by the treating veterinarian or physician. Any appropriate means of administration can be used, also as determined by the treating veterinarian or physician. The treatments can be combined with diagnostic assessment before or after therapy. Thus, the therapy can be personalized to the subject in need of such treatment.

Still further provided is a method of treating a pathogenic condition in a subject in need thereof, comprising, or consisting essentially of, or yet further consisting of administering to the subject an antigen or antigenic peptide joined to the bacteriophage and/or VLP or a composition or a composition as described herein. In one aspect, the pathogen is a virus, e.g., a coronavirus, a human immunodeficiency virus (HIV) or a Hepatitis virus, optionally a Hepatitis B virus or a Hepatitis C virus. In another aspect, the pathogen is a bacterium, protozoan, helminth, prion, or fungus. Non-limiting examples of such include Vibrio parahaemolyticus or rock bream iridovirus, Edwardsiella tarda, or Vibrio vulnificus.

The methods can be practiced clinically, e.g. in a human subject or in laboratory animals as an animal model to test for new combination therapies. Methods to measure pathogenic infection are known in the art.

In one aspect, the target cell or population comprising the target cell comprises a cancer cell, or a cancer or tumor, e.g. a solid tumor. Non-limiting examples of a solid tumors or cells are bladder cancer, bone cancer, brain cancer, breast cancer, colorectal cancer, colon cancer, esophageal cancer, eye cancer, head and neck cancer, kidney cancer, lung cancer, melanoma, ovarian cancer, pancreatic cancer, prostate cancer, or stomach cancer. In a further aspect, the solid tumor is a colon cancer, pancreatic cancer or melanoma. In a further aspect, the cancer or cell is a hematologic malignancy, such as, for example, a lymphoma or leukemia. Non-limiting examples include a B-cell lymphoma, a T-cell lymphoma, a Hodgkin's lymphoma or a non-Hodgkin's lymphoma. The cancer can be primary or metastatic, e.g., Stage I, Stage II, Stage III or Stage IV. It also can be relapsed or refractory cancer. The cell can be a primary cell obtained from example, a biopsy or an established cell line obtained from example a commercial source such as the American Type Culture Collection (ATCC).

In one aspect, the method inhibits the growth of a cancer cell or tumor growth. In one aspect, the method is practiced in vitro and can serve as an assay to test new combination therapies or dosage regimens. It also can be used as a personalized assay by administering to a subject's cancer or tumor cell in vitro the VLP formulation or composition containing same to the cell. One of skill in the art can use conventional assays, such as reduction in tumor size, tumor burden or a reduction in an appropriate cancer marker to determine if the method is appropriate for the subject in need of such treatment or if the treatment has been successful or requires repeating or a change in dosage. The methods can be practiced clinically, e.g. in a human subject or in laboratory animals as an animal model to test for new combination therapies. One of skill in the art can use conventional assays, such as reduction in tumor size, tumor burden or a reduction in an appropriate cancer marker to determine if the method is appropriate for the subject in need of such treatment.

The subject of these methods can be an animal, a mammal or a human in need of such treatment. When the methods are practiced in vitro, the cell can be an animal cell, a mammalian cell or a human cell. In one aspect an effective amount is administered which can be determined using conventional techniques. When the treatment relates to cancer therapy, the method or treatment can be a first-line, second-line, third-line or fourth-line therapy. In can be an adjuvant therapy and combined with other therapies as determined by the treating veterinarian or physician. Any appropriate means of administration can be used, also as determined by the treating veterinarian or physician. The treatments can be combined with diagnostic assessment before or after therapy. Thus, the therapy can be personalized to the subject in need of such treatment.

In another aspect, the target cell or plurality of target cells comprise a cell infected by a pathogen. In one aspect, the pathogen is a virus, e.g., a coronavirus, a human immunodeficiency virus (HIV) or a Hepatitis virus, optionally a Hepatitis B virus or a Hepatitis C virus. In another aspect, the pathogen is a bacterium, protozoan, helminth, prion, or fungus. Non-limiting examples of such include Vibrio parahaemolyticus or rock bream iridovirus, Edwardsiella tarda, or Vibrio vulnificus. The methods can be practiced clinically, e.g. in a human subject or in laboratory animals as an animal model to test for new combination therapies. Methods to measure pathogenic infection are known in the art.

In one aspect, the method is practiced in vitro and can serve as an assay to test new combination therapies or dosage regimens. The methods can be practiced clinically, e.g. in a human subject or in laboratory animals as an animal model to test for new combination therapies. One of skill in the art can use conventional assays, to determine efficacy. The subject of these methods can be an animal, a mammal or a human in need of such treatment.

When the methods are practiced in vitro, the cell can be an animal cell, a mammalian cell or a human cell. In one aspect an effective amount is administered which can be determined using conventional techniques. When the treatment relates to cancer therapy, the method or treatment can be a first-line, second-line, third-line or fourth-line therapy. In can be an adjuvant therapy and combined with other therapies as determined by the treating veterinarian or physician. Any appropriate means of administration can be used, also as determined by the treating veterinarian or physician. The treatments can be combined with diagnostic assessment before or after therapy. Thus, the therapy can be personalized to the subject in need of such treatment.

In some embodiments as described herein, the antigen or antigenic peptide joined to the bacteriophage and/or VLP or a composition as described herein are administered for clinical, e.g. in a human subject or therapeutic applications.

In some embodiments, the an antigen or antigenic peptide joined to the bacteriophage and/or VLP or a composition as described herein are administered to a subject by multiple administration routes, including but not limited to, parenteral, oral, buccal, rectal, sublingual, or transdermal administration routes. In some cases, parenteral administration comprise, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the antigen or antigenic peptide joined to the bacteriophage and/or VLP or a composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously, alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Kits

As used herein, a kit or article of manufacture described herein include a carrier, package, or container that is compartmentalized to receive one or more containers such as vials, tubes, and the like, each of the container(s) comprising, or consisting essentially of, or yet further consisting of, one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Experiment No. 1

Applicant describes herein a drug and vaccine platform capable of both plug-and-play and co-delivery. A nitrilotriacetic acid (NTA) linker conjugated for example to cowpea mosaic virus (CPMV), a plant virus, or virus-like particles (VLPs) from bacteriophage Qβ, through simple lysine, N-hydroxysuccinimide (NHS) chemistry. Both CPMV and Qβ have shown remarkable efficacy as vaccine adjuvants.¹⁶⁻²¹ The NTA group complexes with any histidine (His)-tagged protein of interest in the presence of a nickel (Ni) ion.²² Plug-and-play is achieved as the target antigen can be exchanged if it contains a His-tag. In fact, many recombinant proteins are already His-tagged to aid in the protein purification process.^{23,24} Assuming the His-tag does not alter the function or immunogenicity of the antigen, post-purification cleavage and additional processing would not be required potentially saving time and lowering costs during vaccine formulation.^{23,25}

Traditional protocols for viral nanoparticle vaccine formulations have focused on chemical conjugation. For example, Kentucky Bioprocessing, Inc. creates their SARS-CoV-2 vaccine through chemical conjugation of the receptor binding domain of SARS-CoV-2 onto tobacco mosaic virus.³² However, chemical conjugation can have its fair share of drawbacks. First, conjugation of large protein antigens is difficult, and it must be tailored to the protein of interest, which does not allow for plug-and-play capabilities. Second, chemical conjugation may lead to antigen display in different configurations (i.e., when a protein has multiple conjugation sites). This can lead to batch-to-batch variability and inconsistent immune responses against the target antigen. Lastly, conjugation can lead to epitope masking, aggregation, and disruption of protein structures, which must be experimentally resolved and can take extended periods of time.³³ To overcome this, peptide epitopes of the antigen have been used. These vaccine formulations require simplified bioconjugation/genetic display procedures leading to greater yields, better reproducibility, and increased quality control and assurance. However, with peptide vaccination, the breadth of antibody response becomes quite narrowed leading to limited neutralization³⁴—vaccine ineffectiveness then leads to a complete restart of the vaccine formulation starting from epitope design. The NiNTA: His-tag approach combats these drawbacks: by binding the full-length antigen in a controlled manner through engineered His-tags, Applicant gets simple, non-tailored binding with a broad antibody response to the full-length protein. This design was tested using the CPMV and Qβ adjuvants and the common model antigen ovalbumin (OVA). Applicant tested these vaccines for improved antibody production compared to simple admixtures of OVA and CPMV/Qβ and demonstrate efficacy in a mouse model of OVA-expressing melanoma (B16F10-OVA) in C57BL/6J mice.

The following are provided to illustrate and not limit the invention of this disclosure.

### Experimental Methods

Materials: Potassium phosphate monobasic, potassium phosphate dibasic, and Tween-20 were purchased from Fisher Scientific. Phosphate buffered saline (PBS) was purchased from G Biosciences. Dimethyl sulfoxide (DMSO), OVA, bovine serum albumin (BSA), and nickel (II) chloride hexahydrate were purchased from Sigma-Aldrich. 2-iminothiolane, 3-morpholinopropane-1-sulfonic acid (MOPS) buffer, and Tris acetate EDTA (TAE) buffer were purchased from Thermo Fisher Scientific. Dithiothreitol (DTT) was purchased from Gold Biotechnologies. The His₆-maleimide peptide was purchased from Genscript.

Cells: The B16F10-OVA cell line was a generous gift from Dr. Mary Jo Turk at Dartmouth College. The B16F10-OVA was grown and passaged in RPMI-1640 supplemented with 10% (w/v) fetal bovine serum (FBS)+1% (v/v) penicillin/streptomycin (P/S). RPMI was purchased from Corning, FBS was purchased from R&D Systems, and P/S was purchased from Cytiva. The cells were kept in 5% CO₂ and 37° C.

Preparation of CPMV-NiNTA: His-OVA and Qβ-NiNTA: His-OVA Vaccines: CPMV was propagated in black-eyed pea No. 5 plants and purified as previously reported.³⁵ Qβ VLPs were produced in B121 E. coli (DE3) (New England BioLabs) and purified as previously reported.³⁶ CPMV was stored in 0.1 M potassium phosphate (KP) buffer pH 7.2 while Qβ was stored in 1×PBS pH 7.2. Both virus nanoparticles were stored at 4° C. until further use.

CPMV and Qβ were resuspended in 10 mM KP by buffer exchange using 100 kDa molecular weight cut off (MWCO) spin filters (EMD Millipore). The viral capsids were modified with NTA through the addition of 3000 mol equivalents (eqs) per virus nanoparticle of NTA-PEG2K-NHS (Nanocs) diluted in DMSO and allowed to react overnight (ON) at 4° C.; the final DMSO concentration was kept to a maximum of 10% by volume. Excess NTA-PEG2K-NHS was removed using Sephadex G-25 columns (Cytiva) according to the manufacturer's protocols. Ni (5 mM) was added to the solution and incubated ON at 4° C. before removal of Ni through dialysis ON in 10 mM KP. The resulting samples were stored at 4° C. in 10 mM KP until further use. To ensure the presence of bound Ni, the CPMV-NiNTA sample was incubated with 330 mM of DTT; a brown color change indicates the presence of Ni within the solution.

OVA was chemically His-tagged for binding to Qβ and CPMV. The OVA was resuspended to 10 mg mL-1 in water before the addition of 10 mol eqs of 2-iminothiolane (2 mg mL-1 in deionized (DI) water) per OVA. The reaction was run for 2 hrs followed by removal of excess 2-iminothiolane using 10 kDa MWCO spin filters. 4 mol eqs of a His-tag with an N-terminal maleimide (Genscript, sequence: maleimide-HHHHHHHH or maleimide-His₆) was conjugated to the introduced thiol groups and allowed to react ON at 4° C. Excess His-tag was removed through dialysis using a 12-14 kDa MWCO dialysis membrane, and the OVA-His was stored at 4° C. in DI water until further use.

To create the CPMV-NiNTA: His-OVA or Qβ-NiNTA: His-OVA vaccines, 500 mol eqs of the His-OVA was added per CPMV-NiNTA and Qβ-NiNTA and allowed to react ON at 4° C. The unbound His-OVA was removed with a 100 kDa MWCO dialysis membrane in 10 mM KP, and the resulting CPMV-NiNTA: His-OVA and Qβ-NiNTA: His-OVA were stored at 4° C. in 10 mM KP until further use. The same procedures were carried out using CA and BSA proteins.

### Characterization:

Concentration: The concentration of CPMV-NiNTA: His-OVA was analyzed using UV-VIS (Nanodrop 2000). The absorbance was measured at 260 and 280 nm, and an absorbance ratio of 260 to 280 near 1.8 was used to ascertain unbroken, pure particles. The concentration was measured using Beer's Law and the absorbance value at 260 nm with a path length of 0.1 cm and extinction coefficient of 8.1 mL mg-1 cm-1. The concentration of Qβ-NiNTA: His-OVA was analyzed using a Pierce™ BCA assay (Thermo Scientific) according to the manufacturer's protocols. It is noted that the concentrations determined are estimates because the additional protein displayed will also be measured.

SDS-PAGE: SDS-PAGE was carried out to ensure successful conjugation of the His-tag to the OVA and binding of His-OVA to CPMV/Qβ-NiNTA. 10 μg of sample was loaded with 4×lithium dodecyl sulfate Sample Buffer (Life Technologies). In samples with Qβ, a 10×sample reducing agent (Invitrogen) was also added. The samples were then heated at 95° C. for 5 min before running on a 12% NuPAGE gel (ThermoFisher Scientific) at 200 V, 120 mA, and 25 W in 1×MOPS buffer. The gel was visualized with GelCode™ Blue Safe Protein Stain (ThermoFisher Scientific) according to the manufacturer's instructions. The gel was imaged on an AlphaImager (Protein Simple). The amount of bound OVA was calculated using densitometry analysis on ImageJ.

Western blot (WB): To further ensure successful conjugation of the His-tag to OVA, WBs were carried out against the His-tag. Following SDS-PAGE of the His-OVA, the proteins were transferred onto a nitrocellulose paper (VWR) for 1 hr at 25 V, 160 mA, and 17 W. The paper was blocked with 5% (w/v) milk (RPI) for 1 hr and washed 3× with 1×PBS. An α-His HRP antibody (Biolegend) at 0.5 μg mL-1 in 1×PBS was incubated for 1 hr at RT and washed 3× with 1×PBS. A 3,3’-diaminobenzidine (DAB) substrate was incubated for 5 min before washing away 3× with 1×PBS. The nitrocellulose was then read under the AlphaImager System.

The CPMV-NiNTA: His-OVA sample was also assessed through WB. The protocol was unchanged from before except the samples were incubated with either an «-His HRP antibody (0.5 μg mL-1) or an α-OVA mouse antibody (1:1000 dilution, Novus Biologicals). In the samples bound with α-OVA, the nitrocellulose was washed 3× with 1×PBS followed by the addition of an-mouse goat AF647 antibody (1:1000 dilution, Biolegend) for 1 hr at RT. The unbound secondary antibody was washed away 3× with 1×PBS before imaging on the AlphaImager System.

Agarose gel electrophoresis: Electrophoresis was carried out using 10 μg of CPMV-NiNTA: His-OVA and Qβ-NiNTA: His-OVA and a 1.2% (w/v) agarose gel in 1×TAE buffer. 1 μL of GelRed nucleic acid gel stain (Gold Biotechnologies) was added to the gel before running the gel at 30 min at 120 V and 400 mA. The RNA was first visualized using the AlphaImager using a red filter, and then the protein was visualized by incubating the gel in 0.25% (w/v) Coomassie Blue ON followed by imaging on the AlphaImager under white light.

ELISA: Greiner Bio-One 96-well medium-binding microplates were coated with 100 μL of 10 μg mL-1 of an α-OVA mouse antibody (Novus Biologicals) ON at 4° C. The plate was washed 3× with 100 μL of PBS+0.1% (v/v) Tween-20 (PBST). The CPMV/Qβ-NiNTA: His-OVA and control samples were then added to appropriate wells at 50 μg mL-1 and incubated for 1 hr at RT. The wells were washed 3× with PBST and incubated with 100 μL of an a-CPMV or a-Qβ rabbit antibody (Pacific Immunology) at 10 μg mL-1 for 1 hr at RT. The wells were washed 3× with PBST followed by incubation of an α-rabbit goat HRP antibody (1:5000 dilution, Fisher Scientific) for 1 hr at RT. The plate was washed 3× with PBST, and 100 μL of 1-Step Ultra TMB was added to each well. The TMB was reacted for 2 min followed by the addition of 100 μL of 2N H₂SO₄. The plate was read on a microplate reader (Tecan) at 450 nm. All samples were run in triplicate. The ELISAs were carried out on samples 7 and 28 days following the generation of the vaccines.

TEM: TEM was carried out on Formvar carbon film coated TEM supports with 400-mesh hexagonal copper grids (VWR International). The grids were first incubated with the viruses, which were diluted to 0.1 mg mL-1 in DI water, for 2 min followed by staining with 2% uranyl acetate for 2 min. The images were taken on a Joel 1400 TEM at 80 kV.

CD: CD measurements were carried out on an Aviv model 21D CD spectrometer. OVA was diluted to 0.5 mg mL-1 while the viruses were diluted to 0.3 mg mL-1 in 0.1 M KP buffer. Measurements were taken from 180 to 320 nm at RT at stepwise increments of 1 nm. Readings were taken 2-3 times and averaged.

DLS: The samples were diluted to 0.1 mg mL-1 in DI water before reading on a Zetasizer Nano ZSP/Zen5600 (Malvern Panalytical). The samples were run at 25° C. with a 20 s equilibration time. The OVA-bound samples were measured on days 3, 7, 14, 21, and 28 following the binding.

FPLC: FPLC measurements were taken on an Äkta pure 25 M1 (Cytiva). Samples were diluted to 1 mg mL-1 in 150 μL of 10 mM KP. The flow rate was set to 0.5 mg mL⁻¹ and an isocratic elution profile was used. Absorbance measurements were taken at 260 and 280 nm. FPLC was run on samples 7, 14, 21, and 28 days following the generation of the vaccines.

Mice Immunization: All animal experiments were carried out in accordance with the guidelines set out by the IACUC of the University of California, San Diego. All mice were purchased from Jackson Labs and housed at the Moores Cancer Center. The mice were granted unlimited food and water at all times.

C57BL/6J mice were immunized through 3 injections spaced two weeks apart of CPMV-NiNTA: His-OVA, Qβ-NiNTA: His-OVA, CPMV+OVA, Qβ+OVA, CPMV, Qβ, and OVA. The injections were done subcutaneously (s.c.) and standardized to the OVA concentration (5 μg), which meant that for the CPMV- and Qβ-containing groups, 41 and 25 μg of CPMV/Qβ-NiNTA: His-OVA and control samples were injected, respectively. Mice blood was collected every two weeks through retroorbital (r.o.) bleeding until 6 weeks past the first dose. The sera were isolated through centrifugation of blood at 2,000×g for 10 min at 4° C. and collection of the supernatant. Sera were stored at −80° C. until further use.

Antibody Titer Measurements and Antibody Isotyping: Antigen-specific antibodies were quantified using ELISA. Greiner Bio-One medium-binding 96-well plates were coated with 100 μL of 10 μg mL-1 of OVA in 50 mM carbonate-bicarbonate buffer pH 9.6 ON at 4° C. The plates were washed with PBST 3× and blocked with 1×casein blocking buffer with fish gelatin (Bioworld) in 1×PBS for 1 hr at RT. Following washing, the sera of the mice were added at a starting dilution of 1:200 followed by serial dilutions of 2. The sera were incubated for 1 hr at RT followed by washing. Goat «-mouse HRP IgG secondary antibodies specific to the Fc region were added to the plate and incubated for 1 hr at RT. The secondaries were washed 3× with PBST, and 100 μL of 1-Step Ultra TMB was incubated for 2 min followed by the addition of 100 μL of 2N H₂SO₄. The plate was then read on a microplate reader at 450 nm. The endpoint titer was considered the dilution at which the absorbance of the samples was greater than twice that of the blanks.

The isotype of the antibodies that were produced was also investigated through ELISA. In this case, the sera within each group were pooled and diluted 1:1000. When adding the secondary antibodies, isotype specific antibodies with conjugated HRP were added (IgGₜₒₜₐₗ, IgG1, IgG2b, IgG2c, IgA, IgM, and IgE). All secondaries were added at a dilution of 1:5000 except for IgE, which was diluted 1:1000. The ratio of IgG2b IgG1-1 and IgG2c IgG1-1 was calculated, and a value <1 was considered a Th2 response while >1 was considered Th1. All the secondary antibodies were purchased from Biolegend.

Tumor Inoculation: In the same mice used above for antibody titer measurements, at week 6, 200,000 B16F10-OVA cells were injected s.c. in 200 μL of 1×PBS. The tumors were measured every 2 days, and the survival of the mice was followed. Mice were euthanized when their tumors reached >1500 mm³ with tumor volume measured using the equation: V=l×w²/2.

Statistical Analysis: ELISA data proving binding between OVA and CPMV/Qβ was analyzed using one-way ANOVA. Endpoint titers were analyzed with two-way ANOVA. Tumor volume curves were analyzed with two-way ANOVA while the delay of tumor onset bar graphs were analyzed with one-way ANOVA. All analyses were done on GraphPad Prism.

### Results and Discussion

### Vaccine Production and Characterization

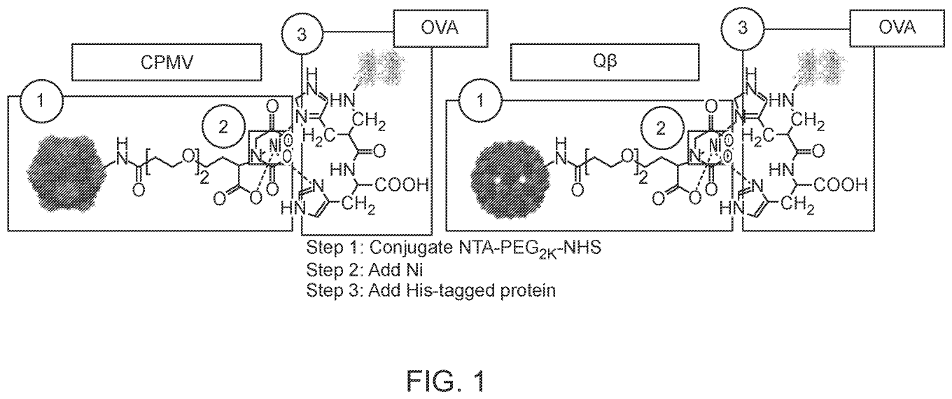

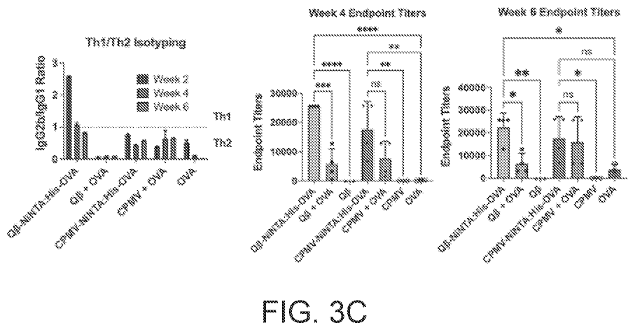

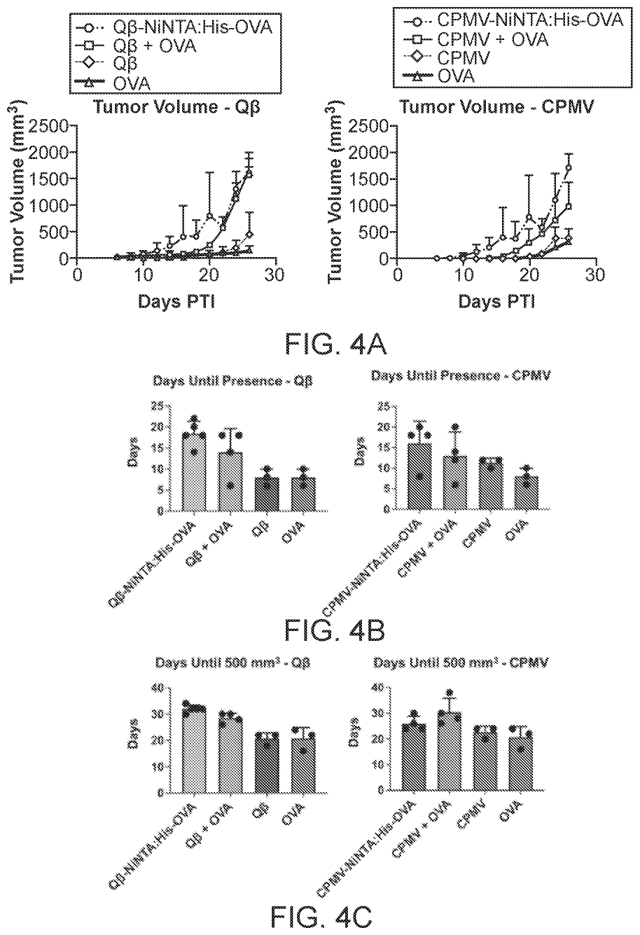

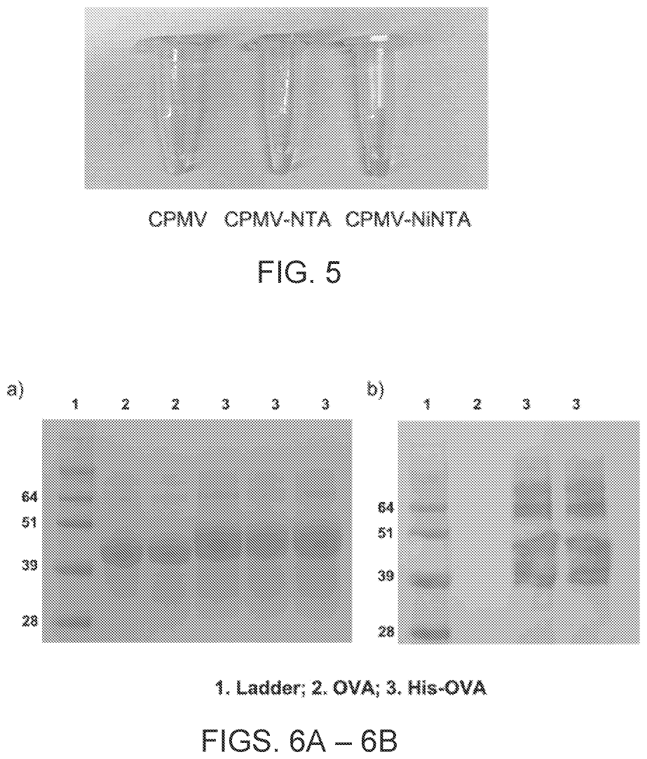

CPMV was harvested from black-eyed pea no. 5 plants while Qβ VLPs were expressed and purified from B121 (DE3) E. coli as previously reported. 35,36 The capsids of CPMV and Qβ both contain external lysines (300 on CPMV,³⁷ 720 on Qβ38)-thus, the exterior lysines on CPMV and Qβ were first conjugated to an NTA-PEG_{2κ}-NHS linker (FIG. 1). The NTA group was then reacted with Ni overnight. Following purification of unbound Ni, Applicant ascertained the presence of bound Ni using dithiothreitol (DTT), which causes a color change of the solution to a light brown color following reduction of Ni ions (FIG. 5).³⁹ The NiNTA group then serves as a linker to bind a His-tagged protein of interest. Proteins are commonly genetically engineered with His-tags for purification.²³ For this experiment, Applicant manually inserted His-tags into native target proteins through bioconjugation (FIG. 6). This was accomplished by reacting 2-iminothiolane to solvent-exposed lysines on the model antigen, OVA, therefore introducing a thiol group to couple to maleimide-terminated His₆ peptides. The successful addition of the His-tag (His₆) by chemical conjugation to OVA was verified by sodium dodecyl sulfate-polyacrylamide gel electrophoresis (SDS-PAGE) and Western blot (WB) (FIG. 6). It is acknowledged that chemical tagging of the protein with a His-tag may not provide orientational control of display, but this approach is highly effective to demonstrate that several His-tagged proteins could be easily obtained through the chemical coupling of the His-tagged peptides (see FIG. 7). This approach demonstrates wide pertinency for a variety of applications due to the plug- and -play nature of the vaccine formulation.

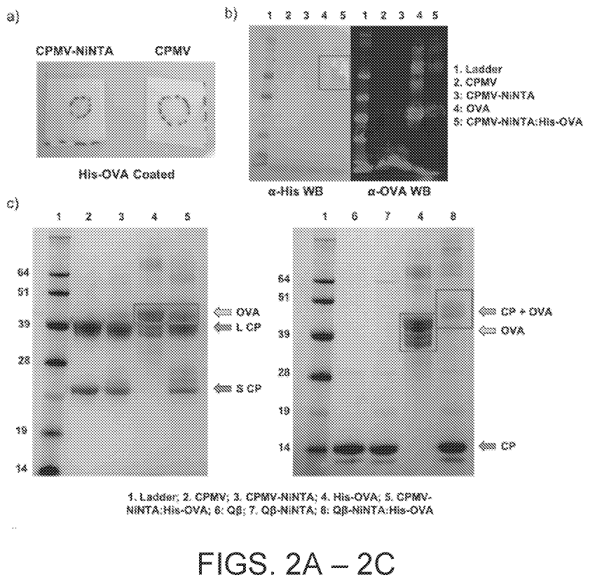

To ensure that the NiNTA-conjugated virus-based nanoparticles and the His-tagged OVA (His-OVA) were indeed coupled with the antigen, dot blots (DBs) were carried out (FIG. 2A). His-OVA was spotted onto a nitrocellulose membrane and then probed with either CPMV-NiNTA or native CPMV. Binding was visualized using an a-CPMV antibody and a horseradish peroxidase (HRP)-conjugated secondary antibody. Binding to His-OVA was only observed using CPMV-NiNTA (FIG. 2A). Next, the complex was formed in solution and purified, followed by characterization using WB and SDS-PAGE (FIG. 2B, FIG. 2C).

In the WB probed with α-His and α-OVA antibodies, His and OVA were successfully detected in the CPMV-NiNTA: His-OVA samples (FIG. 2B, for WB, OVA served as the control). In SDS-PAGE, it should be noted that free His-OVA is observed, not in complex with the CPMV CPs (FIG. 2C). However, it is expected that the Ni-NTA: His-OVA complex dissociates under the denaturing conditions of SDS-PAGE. While Applicant cannot rule out entirely the presence of unbound His-OVA, native gels and ELISA further support the successful formation of the CPMV-NiNTA: His-OVA complex (FIGS. 2D-2G). Somewhat contrasting, in the Qβ-NiNTA: His-OVA samples, the His-OVA remains bound to the Qβ coat protein (CP) as evidenced by the upwards shift of the His-OVA protein band by ˜14 kDa, the molecular weight of one Qβ CP.⁴⁰ While somewhat puzzling, the His-OVA may not release after heating and denaturation from Qβ-NiNTA possibly due to the multivalency and avidity effects because Qβ exhibits a higher density of lysines and hence NiNTA. The amount of NTA bound per particle could not be determined, but there were ˜15 and 13 His-OVA per CPMV and Qβ, respectively. The amount of bound OVA was calculated using densitometry analysis on ImageJ. As mentioned above, CPMV and Qβ contain 300 and 720 addressable lysines on the exterior of the viral capsid.^{37,38} However based on labeling with fluorophores (small molecules) it was expected that ˜100 NTA displayed per nanoparticle; thus Applicant estimate an OVA display efficiency at ˜ 15%. Based on the size of OVA and the available surface area of a 30-nm sized nanoparticle, a densely packed arrangement would equate to ˜39 OVA per nanoparticle.

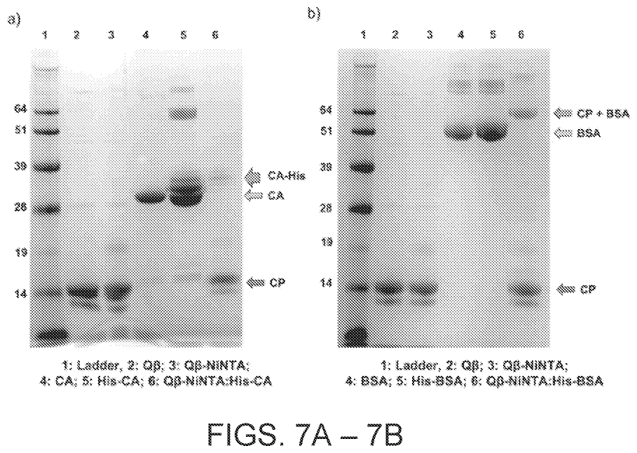

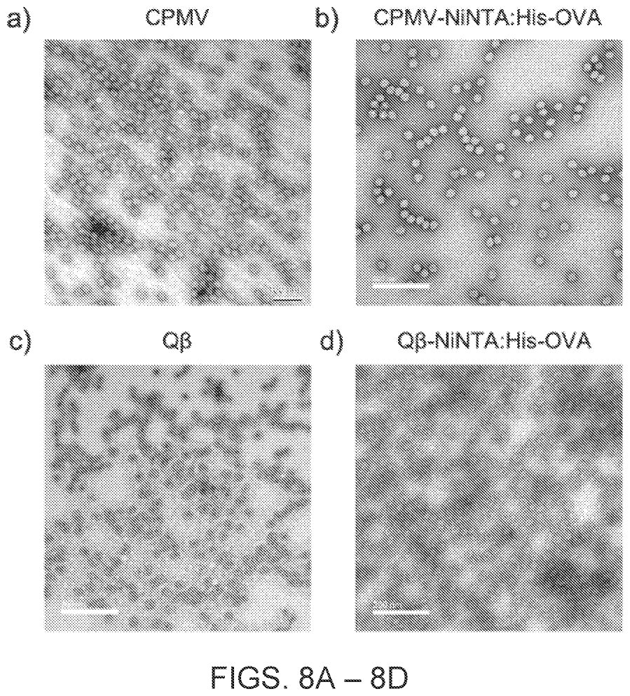

The CPMV-NiNTA: His-OVA and Qβ-NiNTA: His-OVA vaccines were further characterized by agarose gel electrophoresis (FIG. 2D). Changes in molecular weight and charge can influence the electrophoretic mobility. Coupling of the NTA-PEG2K-NHS linker and binding of Ni reduced the mobility of the particles. The binding of His-OVA reduced mobility even further indicating a step-by-step increase in molecular weight. Co-localization of the RNA (genomic RNA for CPMV and host RNA for Qβ) and protein component indicates stable particle formulations.⁴¹ Furthermore, transmission electron microscopy (TEM) and circular dichroism (CD) of both native and OVA-bound viruses confirm structural integrity of the vaccine formulations (FIG. 8, FIG. 9). TEM shows intact 30 nm-sized nanoparticles and binding of OVA did not change the morphology or structure. Consistent with TEM imaging, the overlayed CD spectra show consistency between the native and OVA-bound viruses with minimal differences in the secondary and tertiary structures which can be explained by the OVA protein loading onto the viral nanoparticles.

Modified ELISA protocols were carried out to validate that the His-OVA was indeed binding to CPMV/Qβ-NINTA (FIGS. 2E-2G). The plate was first coated with an a-OVA antibody followed by the addition of CPMV/Qβ-NiNTA: His-OVA and controls, and then probed using a-CPMV/Qβ antibodies. By utilizing two specific antibodies (α-OVA and α-CPMV/Qβ), only samples containing bound OVA and CPMV/Qβ would be able to produce a signal (see schematic in FIG. 2E). While CPMV-NiNTA: His-OVA produced a strong discernible signal, the controls did not produce a significant signal above background (FIG. 2F). Most importantly, there was no indication of binding or association in the CPMV and OVA admixture (CPMV+OVA). Applicant noticed a similar pattern with the Qβ samples (FIG. 2G).

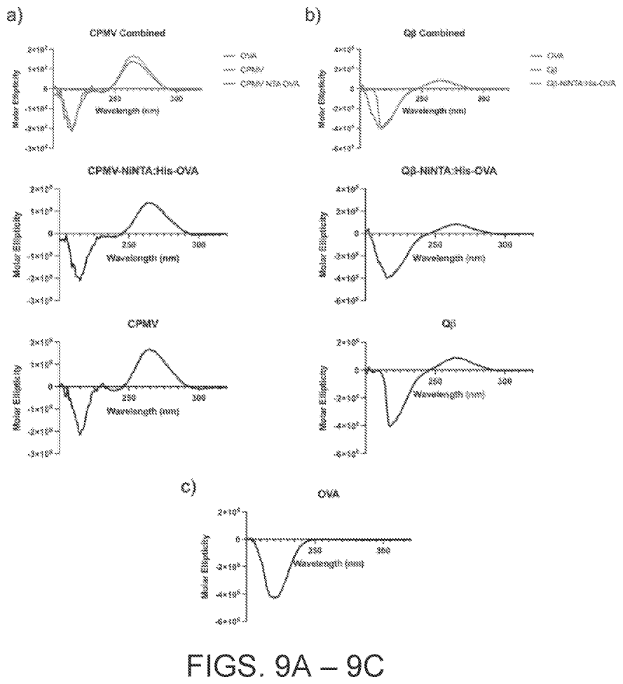

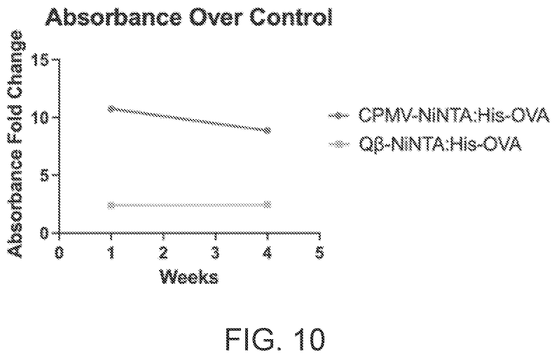

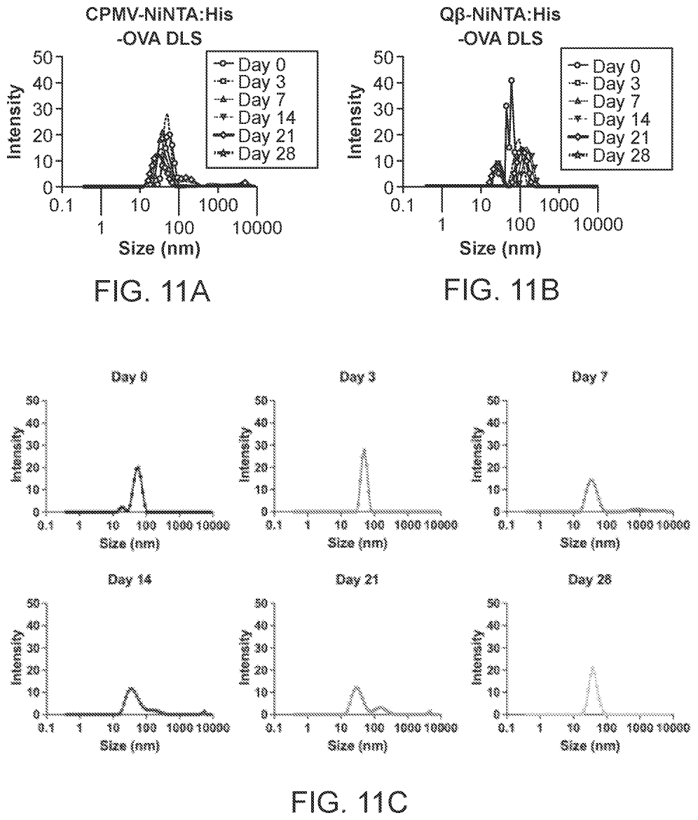

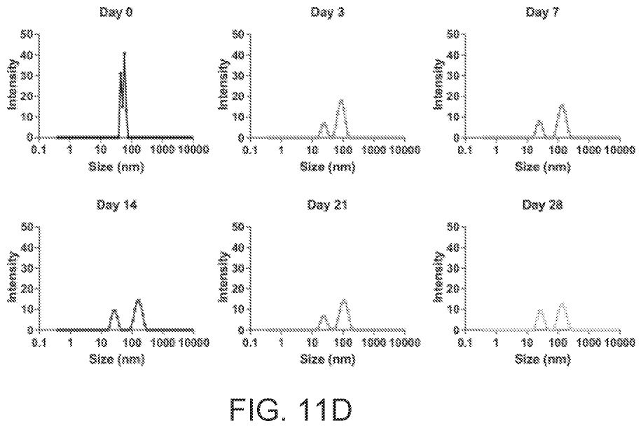

Longitudinal studies utilizing the modified ELISA protocols, fast protein liquid chromatography (FPLC), and dynamic light scattering (DLS) were also carried out to investigate the structural and binding properties of the CPMV/Qβ-NiNTA: His-OVA with respect to time. The ELISAs show that even 4 weeks past the production and assembly of the vaccines, the samples produce multi-fold improvements in absorbance over the controls indicating binding between the CPMV/Qβ and the His-OVA occurs long-term (FIG. 10). The DLS data of CPMV-NiNTA: His-OVA shows minor levels of aggregation starting at day 7 most likely due to the decreased stability of the formulations following OVA disassembly (FIG. 11A, FIG. 11C). The Qβ-NiNTA: His-OVA displayed slightly different properties in that there was almost immediate aggregation of the particles starting 1 day after the vaccines were generated, however, to a much smaller degree compared to the CPMV-NiNTA: His-OVA (FIG. 11B, FIG. 11D). The aggregated particles were in the range of 100-200 nm, and only ˜ 50% of the particles were aggregated. The constant nature of the aggregation most likely indicates an equilibrium of association/dissociation of the OVA had occurred, which is also represented by the similar ELISA levels seen throughout the longitudinal study (FIG. 10). Unlike the DLS data, the FPLC spectra for both the CPMV and Qβ-NiNTA: His-OVA did not show any levels of aggregation and the particles were intact (FIG. 12). Additionally, there was no presence of unbound OVA at any of the timepoints-a control experiment with native CPMV and an equimolar ratio of OVA indicated that if 100% of the OVA was unbound, it could be detected by FPLC (FIG. 13). This indicates that either 1) the OVA remains bound to the CPMV/Qβ-NiNTA or 2) the dissociated OVA is too low in concentration to be detected by FPLC.

### Demonstration of Plug-and-Play Capabilities

To validate that the vaccine formulation strategy indeed could be utilized as plug-and-play candidates for future vaccine applications, the Qβ-NiNTA was also tested to be complexed with other proteins such as bovine serum albumin (BSA) and carbonic anhydrase (CA). Applicant chemically His-tagged both of these proteins and then bound them to the Qβ-NiNTA. SDS-PAGE characterization of the Qβ-NiNTA: His-CA and Qβ-NiNTA: His-BSA (FIG. 7) demonstrates the successful binding of these antigens to Qβ-NiNTA and thereby the modular platforms capability.

SDS-PAGE reveals the presence of His-CA and His-BSA, but the pattern is distinct: His-CA dissociates from the Ni-NTA complex under the SDS-PAGE conditions (FIG. 7A). In contrast, His-BSA remains stably bound as was observed with His-OVA (FIG. 7B, FIG. 2C). Therefore, in addition to avidity effects from multivalent NiNTA display on the Qβ-NiNTA nanoparticles, the charge/hydrophobicity of the target protein may come into play to determine the overall stability of the complex. For the Qβ-NiNTA: His-CA complex that disassembles under SDS-PAGE conditions, Applicant noted that only His-tagged CA and not a mixture of native and His-CA was detectable, which supports that free CA was not present in the complex.

### Mice Immunization

### Mice Immunization and Antibody Titers

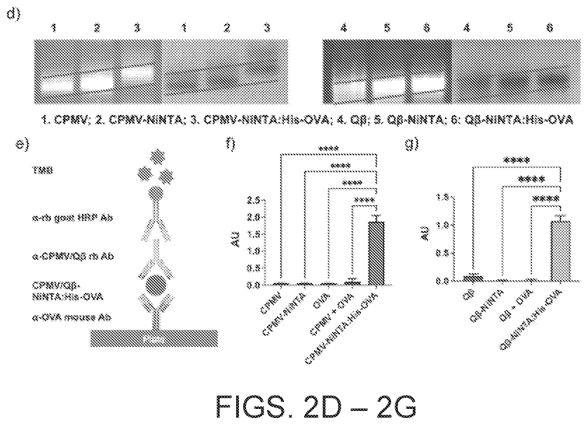

The CPMV/Qβ-NiNTA: His-OVA vaccine formulations were then tested in mice to evaluate effectiveness in generating antibodies against the target antigen, OVA. C57BL/6J mice were immunized using a prime and double-boost regimen spaced two weeks apart (FIG. 3A). Each vaccine was standardized to the OVA concentration with dosing of 5 μg/mouse subcutaneously (s.c.). To estimate the OVA concentration in CPMV-NiNTA: His-OVA vs. Qβ-NiNTA: His-OVA, SDS-PAGE and ImageJ lane analysis was performed. Mice received 41 μg of CPMV or CPMV-NiNTA: His-OVA and 25 μg of Qβ or Qβ-NiNTA: His-OVA. The molecular weight of CPMV is ˜2.25×greater than Qβ, so although similar amounts of OVA were bound to each virus, a greater weight of CPMV was injected. Blood was collected every two weeks until week 6 and antibody titers and subtypes were evaluated using ELISA.

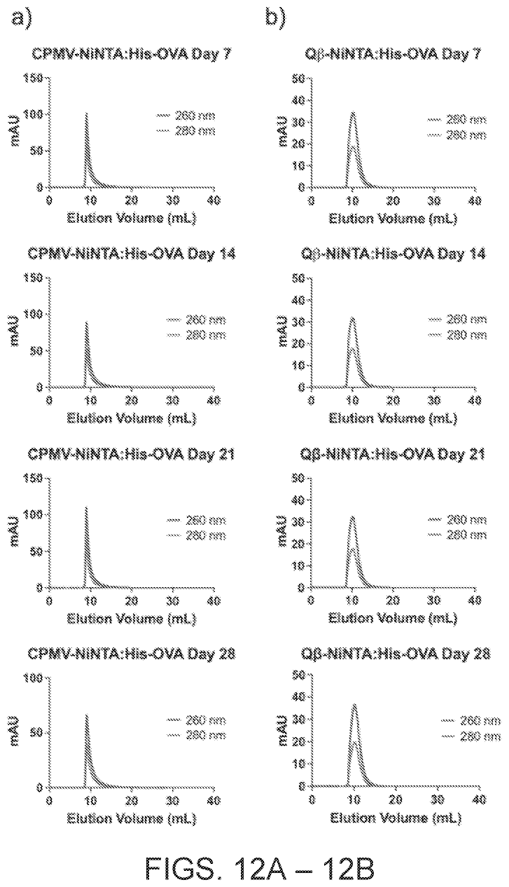

At week 2, the titers are low as expected; however, even at week 2, data indicate a 4.4-fold increase in titers for the Qβ-NiNTA: His-OVA vs. the Qβ+OVA admixture (FIG. 14). By week 4, the Qβ-NiNTA: His-OVA titers were 4.5- and 128-fold that of the admixture (p<0.001) and OVA only control (p<0.0001), respectively (FIG. 3B). At the last measured timepoint (week 6), the Qβ-NiNTA: His-OVA titers remained 2.3- and 6-fold improved compared to the admixture (p<0.05) and OVA (p<0.05), respectively. However, unlike with the Qβ groups, in the CPMV groups, there was no clear difference between the NiNTA vaccine and admixture. At week 4, the CPMV-NiNTA: His-OVA was 2.3- and 88-fold that of the admixture (p>0.05) and OVA only control (p<0.01), respectively. By week 6, the titers between CPMV-NiNTA: His-OVA and CPMV+OVA were identical and notably ˜4.7-fold greater than the OVA only control (p<0.05).

The results demonstrate that with the Qβ formulation, Qβ-NiNTA: His-OVA outperformed the simple admixture of Qβ+OVA in terms of antibody production against the target protein OVA. This may be explained by the fact that OVA is being co-delivered with Qβ in the Qβ-NiNTA: His-OVA formulation, therefore achieving co-delivery of antigen and adjuvant to the same cell.⁴²⁻⁴⁴

With CPMV there were no clear differences between the CPMV-NiNTA: His-OVA and CPMV+OVA formulations which may indicate that (i) not all viral adjuvants may require co-delivery to achieve potency, (ii) the complex had dissociated, as observed in the SDS-PAGE (see FIG. 2C), or (iii) that complexation in media led to co-delivery even with the admixture. In fact, it was recently shown that proteins may adsorb onto plant viral adjuvants even without integrated complexation chemistry and that some plant viral adjuvants remain efficacious without complexation.^{45,46} The data demonstrates that whether or not an antigen binds is a function of the protein chemistry of the antigen and viral adjuvant. For future experiments, one could utilize a trivalent NTA as opposed to the monovalent one used here to improve the binding kinetics between CPMV and OVA (monovalent NTA has a K_{d} of ˜10 μM and trivalent NTA has a K_{d} of ˜1 nM).^{47,48} The data also indicates that OVA by itself can elicit titers after 3 total injections, but that an adjuvant like CPMV or Qβ greatly improves titer production.

Lastly, Applicant does concede that conjugation of OVA to the viruses would most likely boost antibody response compared to the NTA: His chemistry as the OVA would not dissociate in vivo—others have indeed showed that conjugation provides the best antibody response.⁴⁷ However, conjugation can be difficult, and in our own experiments, conjugation of OVA to our virus particles utilizing both EDC/NHS and NHS maleimide chemistry were unsuccessful (data not shown) providing further evidence that for rapid development of vaccine candidates, a non-tailored approach such as with the NTA: His can greatly improve the development speed.

### Antibody Isotyping

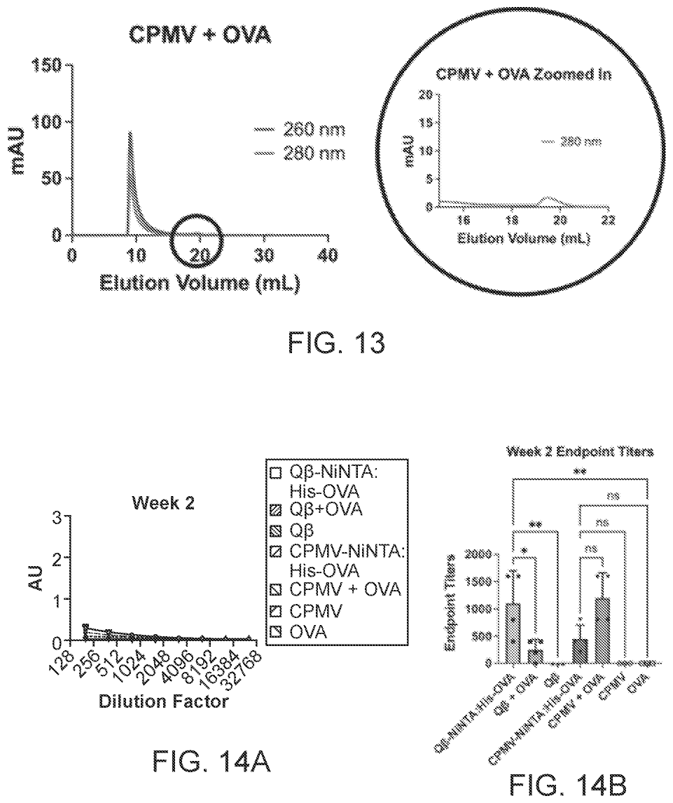

The antibodies were further investigated for their IgG isotypes as well as any other Ig subtypes. A ratio of IgG2b IgG1⁻¹<1 is seen as a Th2 balance while a ratio >1 is Th1.⁴⁹ In the Qβ-NiNTA: His-OVA group, the bias skewed strongly Th1 at week 2 and then moved to a balanced Th1/Th2 bias starting from week 4 and remained balanced at week 6 (FIG. 3C). All the other groups (Qβ+OVA, CPMV-NiNTA: His-OVA, CPMV+OVA, and OVA) skewed strongly Th2 starting from week 2 and remained Th2 biased. The difference in bias between the Qβ-NiNTA: His-OVA and Qβ+OVA indicates that even with the same adjuvant/antigen combination, co-delivery can vastly affect the types of antibodies that are produced. When looking at the other antibody isotypes, all the vaccine groups were able to produce small quantities of IgM, as part of the onset of antibody production and IgM-to-IgG switch (FIG. 15).⁵⁰ Other antibody classes were not detected. Of note, IgE, which is known to elicit allergic responses, was not detected indicating the safety of the vaccines.

For cancer vaccines, generally a Th1 bias is desired, as this promotes cytotoxic T cell priming and destruction of cancer cells with increased safety when targeting self-antigens.⁵¹ Alternatively, active immunization to generate therapeutic antibodies (which is Th2-mediated) also has shown success, for instance, against HER2-positive cancers.^{52,53} Prior work has shown that CPMV and peptide epitopes have generally indicated that CPMV vaccination induces a strong Th1 bias.^{54-56,36} However in complex with OVA, immunization promotes Th2 bias-therefore, it appears that the T helper cell bias is directly affected by the antigen, vaccine formulation (e.g. implant, microneedle, or bolus injection), and the adjuvant, and the bias can be determined for each antigen/adjuvant combination, using methods known in the art.^{54-56, 36} For example, this can be determined using an ELISA or an ELISPOT to determine the Th1/Th2 bias. For the ELISA method, the sera is run using ELISA with antibodies specific for IgG1, IgG2b, IgG2c as the secondary antibody. The absorbance measurements are calculated from the ELISA, and a ratio of IgG2b/IgG1<1 or IgG2c/IgG1<1 is considered Th2 while a ratio of IgG2b/IgG1 or IgG2c/IgG1>1 is considered Th1. One of skill in the art can use ELISPOT according to the manufacturer's instructions (https://pubs.rsc.org/en/content/articlelanding/2023/TB/D2TB02355E, last accessed on Mar. 20, 2023). In brief, IFNy production by the assay, which is measured by the designated red spots, signifies a Th1 response while the IL-4, measured by other blue spots, signifies a Th2 response.

### Tumor Challenge and Vaccine Efficacy

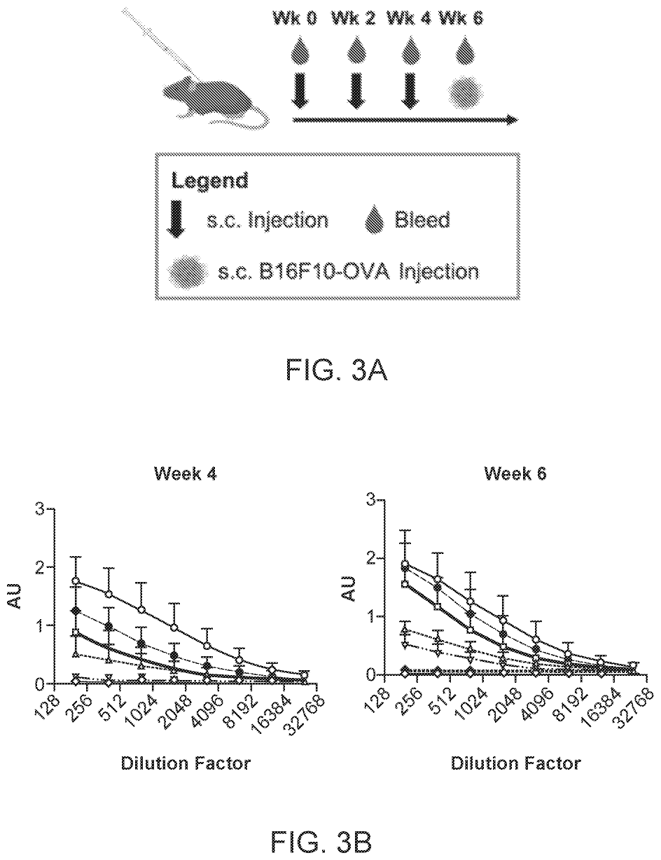

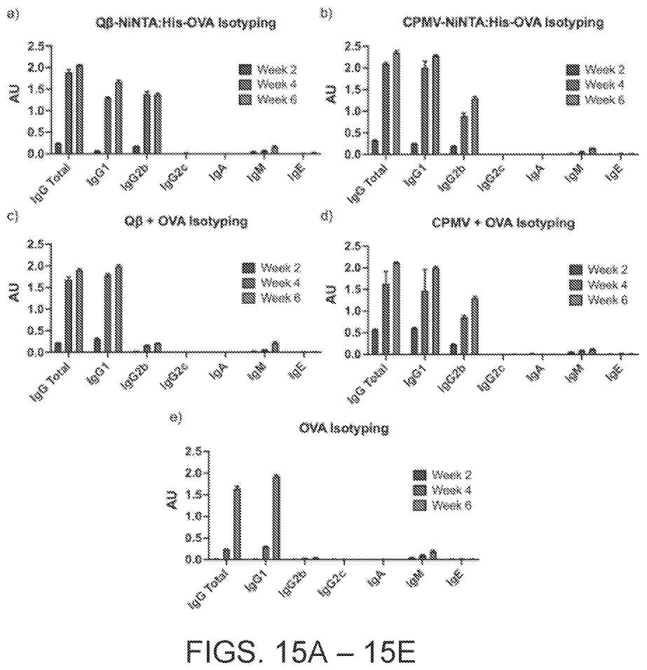

The same mice from above were challenged at week 6 post-immunization with 200,000 B16F10-OVA cells s.c. to determine whether α-OVA antibodies exhibited a therapeutic effect. Indeed efficacy was observed, in particular for the Qβ-NiNTA: His-OVA as well as the Qβ+OVA groups, with Qβ-NiNTA: His-OVA being the most potent formulation significantly reducing tumor burden. On day 20, the average tumor volume was 28.48 and 70.54 mm³ for Qβ-NiNTA: His-OVA and Qβ+OVA treated animals (FIG. 4A). In comparison, the Qβ- and OVA-treated control groups displayed average tumor volumes of 247.5 and 799 mm³. By day 26, the difference in tumor volume between Qβ-NiNTA: His-OVA and Qβ+OVA became more pronounced as Qβ-NiNTA: His-OVA had a 3-fold smaller tumor volume than Qβ+OVA. In the CPMV vaccine groups, the CPMV-NiNTA: His-OVA and CPMV+OVA mice had tumor volumes of 52.4 and 56 mm³, respectively (FIG. 4A). The CPMV only control had a tumor volume of 319.8 mm³, a 6.1- and 5.7-fold difference, respectively. Efficacy data are in good agreement with the antibody titers (see FIG. 3): Qβ-NiNTA: His-OVA produced more α-OVA antibodies vs. the Qβ+OVA admixture and is the more potent vaccine formulation. In contrast, the CPMV-NiNTA: His-OVA and CPMV+OVA groups demonstrated similar antibody production, which was reflected by the tumor rejection. It is important to note that the CPMV and Qβ experiments were run at the same time, which is why they both show identical OVA tumor volumes. They were split into two groups for ease of viewing. The tumor volume curves for all groups can be found in FIG. 16A.

Efficacy is not only apparent by reduced tumor burden, but also by delayed onset of tumor growth. Therefore, Applicant also analyzed how many days passed until tumors were palpable and then reached a size of 500 mm³. For Qβ-NiNTA: His-OVA, it was 18.4 days until tumors were palpable (FIG. 4B). This was 1.3-, 2.3-, and 2.3-fold slower than Qβ+OVA (p>0.05), Qβ (p<0.05), and OVA (p<0.05), respectively. When measuring the days it took tumors to reach 500 mm³, Qβ-NiNTA: His-OVA took 1.1-, 1.5-, and 1.5-fold greater time than Qβ+OVA (p>0.05), Qβ (p<0.01), and OVA (p<0.01), respectively (FIG. 4C). While CPMV-NiNTA: His-OVA fared better than the negative controls, it was in line with the CPMV+OVA group (FIGS. 4B, 4C). The bar graph displaying all groups simultaneously can be found in FIG. 16B, FIG. 16C.

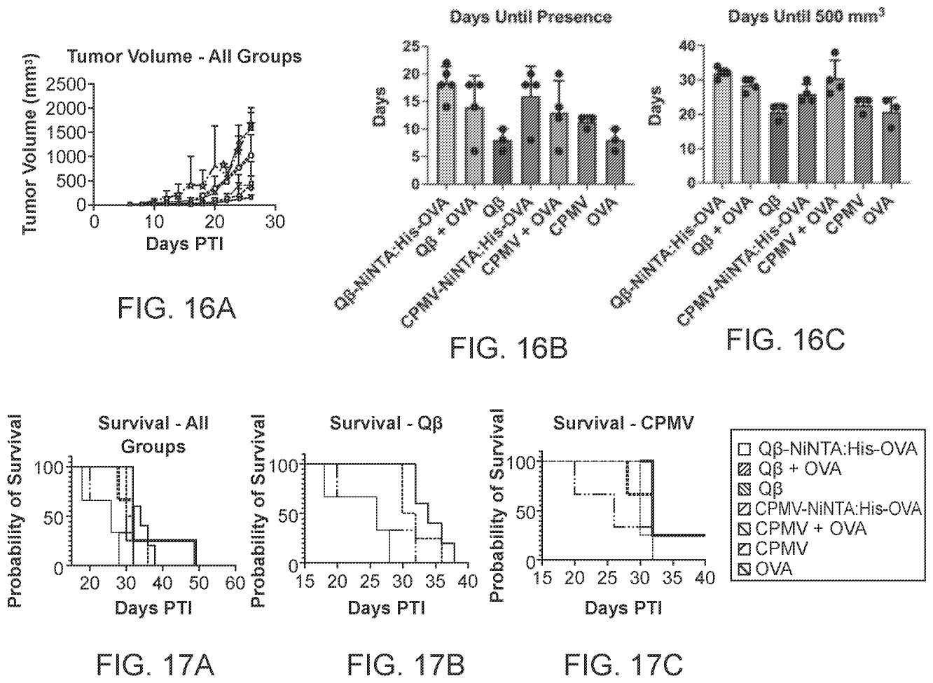

The mice were also measured for survival and were sacrificed at a tumor volume endpoint of 1,500 mm³ (FIG. 17). Qβ-NiNTA: His-OVA and Qβ+OVA improved survival compared to Qβ and OVA with a median survival of 34 and 31 days compared to 26 and 26 days, respectively. Survival was not extended with the CPMV vaccine groups compared to the negative controls.

### Experimental Discussion

Applicant developed and validated a modular vaccine platform making use of plant viral and VLP adjuvant nanoparticles displaying NiNTA for binding of His-tagged antigens. Applicant demonstrate the modularity of this platform by binding OVA as well as other model antigens allowing for a plug-and-play approach for the generation of future vaccines. Applicant utilized the OVA vaccine formulations and demonstrated efficacy in a tumor model using OVA-expressing melanoma cells (B16F10-OVA). Antibody titers and efficacy (reduction of tumor burden/delayed onset of tumor growth) were mirrored demonstrating that Qβ-NiNTA: His-OVA was the most potent formulation outperforming the Qβ+OVA admixture. In contrast, α-OVA antibodies and antitumor efficacy were comparable between the CPMV-NiNTA: His-OVA vs. CPMV+OVA admixture group.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

(1) Bok, K.; Sitar, S.; Graham, B. S.; Mascola, J. R. Accelerated COVID-19 Vaccine Development: Milestones, Lessons, and Prospects. Immunity 2021, 54 (8), 1636-1651. https://doi.org/10.1016/j.immuni.2021.07.017.
(2) Kashte, S.; Gulbake, A.; El-Amin Iii, S. F.; Gupta, A. COVID-19 Vaccines: Rapid Development, Implications, Challenges and Future Prospects. Hum Cell 2021, 34 (3), 711-733. https://doi.org/10.1007/s13577-021-00512-4.
(3) Shin, M. D.; Shukla, S.; Chung, Y. H.; Beiss, V.; Chan, S. K.; Ortega-Rivera, O. A.; Wirth, D. M.; Chen, A.; Sack, M.; Pokorski, J. K.; Steinmetz, N. F. COVID-19 Vaccine Development and a Potential Nanomaterial Path Forward. Nature Nanotechnology 2020, 1-10. https://doi.org/10.1038/s41565-020-0737-y.
(4) Cafri, G.; Gartner, J. J.; Zaks, T.; Hopson, K.; Levin, N.; Paria, B. C.; Parkhurst, M. R.; Yossef, R.; Lowery, F. J.; Jafferji, M. S.; Prickett, T. D.; Goff, S. L.; McGowan, C. T.; Seitter, S.; Shindorf, M. L.; Parikh, A.; Chatani, P. D.; Robbins, P. F.; Rosenberg, S. A. MRNA Vaccine-Induced Neoantigen-Specific T Cell Immunity in Patients with Gastrointestinal Cancer. J Clin Invest 2020, 130 (11), 5976-5988. https://doi.org/10.1172/JCI134915.
(5) Beck, J. D.; Reidenbach, D.; Salomon, N.; Sahin, U.; Türeci, Ö.; Vormehr, M.; Kranz, L. M. MRNA Therapeutics in Cancer Immunotherapy. Mol Cancer 2021, 20 (1), 69. https://doi.org/10.1186/s12943-021-01348-0.
(6) Baden, L. R.; El Sahly, H. M.; Essink, B.; Kotloff, K.; Frey, S.; Novak, R.; Diemert, D.; Spector, S. A.; Rouphael, N.; Creech, C. B.; McGettigan, J.; Khetan, S.; Segall, N.; Solis, J.; Brosz, A.; Fierro, C.; Schwartz, H.; Neuzil, K.; Corey, L.; Gilbert, P.; Janes, H.; Follmann, D.; Marovich, M.; Mascola, J.; Polakowski, L.; Ledgerwood, J.; Graham, B. S.; Bennett, H.; Pajon, R.; Knightly, C.; Leav, B.; Deng, W.; Zhou, H.; Han, S.; Ivarsson, M.; Miller, J.; Zaks, T.; COVE Study Group. Efficacy and Safety of the MRNA-1273 SARS-CoV-2 Vaccine. N Engl J Med 2021, 384 (5), 403-416. https://doi.org/10.1056/NEJMoa2035389.
(7) Polack, F. P.; Thomas, S. J.; Kitchin, N.; Absalon, J.; Gurtman, A.; Lockhart, S.; Perez, J. L.; Pérez Marc, G.; Moreira, E. D.; Zerbini, C.; Bailey, R.; Swanson, K. A.; Roychoudhury, S.; Koury, K.; Li, P.; Kalina, W. V.; Cooper, D.; Frenck, R. W.; Hammitt, L. L.; Türeci, Ö.; Nell, H.; Schaefer, A.; Ünal, S.; Tresnan, D. B.; Mather, S.; Dormitzer, P. R.; Şahin, U.; Jansen, K. U.; Gruber, W. C.; C4591001 Clinical Trial Group. Safety and Efficacy of the BNT162b2 MRNA Covid-19 Vaccine. N Engl J Med 2020, 383 (27), 2603-2615. https://doi.org/10.1056/NEJMoa2034577.
(8) Chung, Y. H.; Beiss, V.; Fiering, S. N.; Steinmetz, N. F. COVID-19 Vaccine Frontrunners and Their Nanotechnology Design. ACS Nano 2020, 14 (10), 12522-12537. https://doi.org/10.1021/acsnano.0c07197.
(9) Borah, P.; Deb, P. K.; Al-Shar′i, N. A.; Dahabiyeh, L. A.; Venugopala, K. N.; Singh, V.; Shinu, P.; Hussain, S.; Deka, S.; Chandrasekaran, B.; Jaradat, D. M. M. Perspectives on RNA Vaccine Candidates for COVID-19. Frontiers in Molecular Biosciences 2021, 8.
(10) Wang, Z.-B.; Xu, J. Better Adjuvants for Better Vaccines: Progress in Adjuvant Delivery Systems, Modifications, and Adjuvant-Antigen Codelivery. Vaccines (Basel) 2020, 8 (1). https://doi.org/10.3390/vaccines8010128.
(11) Kreutz, M.; Giquel, B.; Hu, Q.; Abuknesha, R.; Uematsu, S.; Akira, S.; Nestle, F. O.; Diebold, S. S. Antibody-Antigen-Adjuvant Conjugates Enable Co-Delivery of Antigen and Adjuvant to Dendritic Cells in Cis but Only Have Partial Targeting Specificity. PLOS ONE 2012, 7 (7), e40208. https://doi.org/10.1371/journal.pone.0040208.
(12) Wortham, C.; Grinberg, L.; Kaslow, D. C.; Briles, D. E.; McDaniel, L. S.; Lees, A.; Flora, M.; Snapper, C. M.; Mond, J. J. Enhanced Protective Antibody Responses to PspA after Intranasal or Subcutaneous Injections of PspA Genetically Fused to Granulocyte-Macrophage Colony-Stimulating Factor or Interleukin-2. Infect Immun 1998, 66 (4), 1513-1520. https://doi.org/10.1128/IAI.66.4.1513-1520.1998.
(13) Huang, Q.; Yu, W.; Hu, T. Potent Antigen-Adjuvant Delivery System by Conjugation of Mycobacterium Tuberculosis Ag85B-HspX Fusion Protein with Arabinogalactan-Poly(I: C) Conjugate. Bioconjug Chem 2016, 27 (4), 1165-1174. https://doi.org/10.1021/acs.bioconjchem.6b00116.
(14) Li, W. M.; Dragowska, W. H.; Bally, M. B.; Schutze-Redelmeier, M. P. Effective Induction of CD8+T-Cell Response Using CpG Oligodeoxynucleotides and HER-2/Neu-Derived Peptide Co-Encapsulated in Liposomes. Vaccine 2003, 21 (23), 3319-3329. https://doi.org/10.1016/s0264-410×(03) 00172-5.
(15) Fang, R.; Qiao, S.; Liu, Y.; Meng, Q.; Chen, X.; Song, B.; Hou, X.; Tian, W. Sustained Co-Delivery of BIO and IGF-1 by a Novel Hybrid Hydrogel System to Stimulate Endogenous Cardiac Repair in Myocardial Infarcted Rat Hearts. Int J Nanomedicine 2015, 10, 4691-4703. https://doi.org/10.2147/IJN.S81451.
(16) Nkanga, C. I.; Ortega-Rivera, O. A.; Shin, M. D.; Moreno-Gonzalez, M. A.; Steinmetz, N. F. Injectable Slow-Release Hydrogel Formulation of a Plant Virus-Based COVID-19 Vaccine Candidate. Biomacromolecules 2022, 23 (4), 1812-1825. https://doi.org/10.1021/acs.biomac.2c00112.
(17) Ray, S.; Wirth, D. M.; Ortega-Rivera, O. A.; Steinmetz, N. F.; Pokorski, J. K. Dissolving Microneedle Delivery of a Prophylactic HPV Vaccine. Biomacromolecules 2022, 23 (3), 903-912. https://doi.org/10.1021/acs.biomac.1c01345.
(18) Ortega-Rivera, O. A.; Shukla, S.; Shin, M. D.; Chen, A.; Beiss, V.; Moreno-Gonzalez, M. A.; Zheng, Y.; Clark, A. E.; Carlin, A. F.; Pokorski, J. K.; Steinmetz, N. F. Cowpea Mosaic Virus Nanoparticle Vaccine Candidates Displaying Peptide Epitopes Can Neutralize the Severe Acute Respiratory Syndrome Coronavirus. ACS Infect. Dis. 2021, 7 (11), 3096-3110. https://doi.org/10.1021/acsinfecdis.1c00410.
(19) Ortega-Rivera, O. A.; Shin, M. D.; Chen, A.; Beiss, V.; Moreno-Gonzalez, M. A.; Lopez-Ramirez, M. A.; Reynoso, M.; Wang, H.; Hurst, B. L.; Wang, J.; Pokorski, J. K.; Steinmetz, N. F. Trivalent Subunit Vaccine Candidates for COVID-19 and Their Delivery Devices. J. Am. Chem. Soc. 2021, 143 (36), 14748-14765. https://doi.org/10.1021/jacs.1c06600.
(20) Ortega-Rivera, O. A.; Pokorski, J. K.; Steinmetz, N. F. A Single-Dose, Implant-Based, Trivalent Virus-like Particle Vaccine against “Cholesterol Checkpoint” Proteins. Advanced Therapeutics 2021, 4 (8), 2100014. https://doi.org/10.1002/adtp.202100014.
(21) Lizotte, P. H.; Wen, A. M.; Sheen, M. R.; Fields, J.; Rojanasopondist, P.; Steinmetz, N. F.; Fiering, S. In Situ Vaccination with Cowpea Mosaic Virus Nanoparticles
Suppresses Metastatic Cancer. Nature Nanotech 2016, 11 (3), 295-303. https://doi.org/10.1038/nnano.2015.292.
(22) Knecht, S.; Ricklin, D.; Eberle, A. N.; Ernst, B. Oligohis-Tags: Mechanisms of Binding to Ni2+-NTA Surfaces. J Mol Recognit 2009, 22 (4), 270-279. https://doi.org/10.1002/jmr.941.
(23) Spriestersbach, A.; Kubicek, J.; Schäfer, F.; Block, H.; Maertens, B. Purification of His-Tagged Proteins. Methods Enzymol 2015, 559, 1-15. https://doi.org/10.1016/bs.mie.2014.11.003.
(24) Andersen, K. R.; Leksa, N. C.; Schwartz, T. U. Optimized E. Coli Expression Strain LOBSTR Eliminates Common Contaminants from His-Tag Purification. Proteins 2013, 81 (11), 1857-1861. https://doi.org/10.1002/prot.24364.
(25) Abdullah, N.; Chase, H. A. Removal of Poly-Histidine Fusion Tags from Recombinant Proteins Purified by Expanded Bed Adsorption. Biotechnol Bioeng 2005, 92 (4), 501-513. https://doi.org/10.1002/bit.20633.
(26) Mohsen, M. O.; Augusto, G.; Bachmann, M. F. The 3Ds in Virus-like Particle Based-Vaccines: “Design, Delivery and Dynamics.” Immunol Rev 2020, 296 (1), 155-168. https://doi.org/10.1111/imr.12863.
(27) Chung, Y. H.; Church, D.; Koellhoffer, E. C.; Osota, E.; Shukla, S.; Rybicki, E. P.; Pokorski, J. K.; Steinmetz, N. F. Integrating Plant Molecular Farming and Materials Research for Next-Generation Vaccines. Nat Rev Mater 2021, 1-17. https://doi.org/10.1038/s41578-021-00399-5.
(28) Spohn, G.; Guler, R.; Johansen, P.; Keller, I.; Jacobs, M.; Beck, M.; Rohner, F.; Bauer, M.; Dietmeier, K.; Kündig, T. M.; Jennings, G. T.; Brombacher, F.; Bachmann, M. F. A Virus-Like Particle-Based Vaccine Selectively Targeting Soluble TNF-α Protects from Arthritis without Inducing Reactivation of Latent Tuberculosis. The Journal of Immunology 2007, 178 (11), 7450-7457. https://doi.org/10.4049/jimmunol.178.11.7450.
(29) Bachmann, M. F.; Dyer, M. R. Therapeutic Vaccination for Chronic Diseases: A New Class of Drugs in Sight. Nat Rev Drug Discov 2004, 3 (1), 81-88. https://doi.org/10.1038/nrd1284.
(30) Cubas, R.; Zhang, S.; Kwon, S.; Sevick-Muraca, E. M.; Li, M.; Chen, C.; Yao, Q. Virus-like Particle (VLP) Lymphatic Trafficking and Immune Response Generation after Immunization by Different Routes. J Immunother 2009, 32 (2), 118-128. https://doi.org/10.1097/CJI.0b013e31818f13c4.
(31) Singh, P.; Prasuhn, D.; Yeh, R. M.; Destito, G.; Rae, C. S.; Osborn, K.; Finn, M. G.; Manchester, M. Bio-Distribution, Toxicity and Pathology of Cowpea Mosaic Virus Nanoparticles in Vivo. J Control Release 2007, 120 (1-2), 41-50. https://doi.org/10.1016/j.jconrel.2007.04.003.
(32) Maharjan, P. M.; Choe, S. Plant-Based COVID-19 Vaccines: Current Status, Design, and Development Strategies of Candidate Vaccines. Vaccines (Basel) 2021, 9 (9), 992. https://doi.org/10.3390/vaccines9090992.
(33) Spicer, C. D.; Jumeaux, C.; Gupta, B.; Stevens, M. M. Peptide and Protein Nanoparticle Conjugates: Versatile Platforms for Biomedical Applications. Chem Soc Rev 2018, 47 (10), 3574-3620. https://doi.org/10.1039/c7cs00877e.
(34) Li, W.; Joshi, M. D.; Singhania, S.; Ramsey, K. H.; Murthy, A. K. Peptide Vaccine: Progress and Challenges. Vaccines (Basel) 2014, 2 (3), 515-536. https://doi.org/10.3390/vaccines2030515.
(35) Wen, A. M.; Lee, K. L.; Yildiz, I.; Bruckman, M. A.; Shukla, S.; Steinmetz, N. F. Viral Nanoparticles for In Vivo Tumor Imaging. J Vis Exp 2012, No. 69. https://doi.org/10.3791/4352.
(36) Ortega-Rivera, O. A.; Shin, M. D.; Chen, A.; Beiss, V.; Moreno-Gonzalez, M. A.; Lopez-Ramirez, M. A.; Reynoso, M.; Wang, H.; Hurst, B. L.; Wang, J.; Pokorski, J. K.; Steinmetz, N. F. Trivalent Subunit Vaccine Candidates for COVID-19 and Their Delivery Devices. J. Am. Chem. Soc. 2021, 143 (36), 14748-14765. https://doi.org/10.1021/jacs.1c06600.
(37) Shukla, S.; Wang, C.; Beiss, V.; Cai, H.; Washington, T.; Murray, A. A.; Gong, X.; Zhao, Z.; Masarapu, H.; Zlotnick, A.; Fiering, S.; Steinmetz, N. F. The Unique Potency of Cowpea Mosaic Virus (CPMV) in Situ Cancer Vaccine. Biomater Sci 2020, 8 (19), 5489-5503. https://doi.org/10.1039/d0bm01219j.
(38) Pokorski, D. J. K.; Hovlid, M. L.; Finn, P. M. G. Cell Targeting with Hybrid Qβ Virus-Like Particles Displaying Epidermal Growth Factor. Chembiochem: a European journal of chemical biology 2011, 12 (16), 2441. https://doi.org/10.1002/cbic.201100469.
(39) Le, D. H. T.; Commandeur, U.; Steinmetz, N. F. Presentation and Delivery of Tumor Necrosis Factor-Related Apoptosis-Inducing Ligand via Elongated Plant Viral Nanoparticle Enhances Antitumor Efficacy. ACS Nano 2019, 13 (2), 2501-2510. https://doi.org/10.1021/acsnano.8b09462.
(40) Gorzelnik, K. V.; Cui, Z.; Reed, C. A.; Jakana, J.; Young, R.; Zhang, J. Asymmetric Cryo-EM Structure of the Canonical Allolevivirus Qβ Reveals a Single Maturation Protein and the Genomic SsRNA in Situ. Proceedings of the National Academy of Sciences 2016, 113 (41), 11519-11524. https://doi.org/10.1073/pnas. 1609482113.
(41) Shahrivarkevishahi, A.; Luzuriaga, M. A.; Herbert, F. C.; Tumac, A. C.; Brohlin, O. R.; Wijesundara, Y. H.; Adlooru, A. V.; Benjamin, C.; Lee, H.; Parsamian, P.; Gadhvi, J.; De Nisco, N. J.; Gassensmith, J. J. PhotothermalPhage: A Virus-Based Photothermal Therapeutic Agent. J. Am. Chem. Soc. 2021, 143 (40), 16428-16438. https://doi.org/10.1021/jacs.1c05090.
(42) Hou, Y.; Wang, Y.; Tang, Y.; Zhou, Z.; Tan, L.; Gong, T.; Zhang, L.; Sun, X. Co-Delivery of Antigen and Dual Adjuvants by Aluminum Hydroxide Nanoparticles for Enhanced Immune Responses. Journal of Controlled Release 2020, 326, 120-130. https://doi.org/10.1016/j.jconrel.2020.06.021.
(43) Dong, X.; Liang, J.; Yang, A.; Qian, Z.; Kong, D.; Lv, F. A Visible Codelivery Nanovaccine of Antigen and Adjuvant with Self-Carrier for Cancer Immunotherapy. ACS Appl. Mater. Interfaces 2019, 11 (5), 4876-4888. https://doi.org/10.1021/acsami.8b20364.
(44) Lim, J.-W.; Na, W.; Kim, H.-O.; Yeom, M.; Kang, A.; Park, G.; Park, C.; Ki, J.; Lee, S.; Jung, B.; Jeong, H. H.; Park, D.; Song, D.; Haam, S. Co-Delivery of Antigens and Immunostimulants via a Polymersome for Improvement of Antigen-Specific Immune Response. J. Mater. Chem. B 2020, 8 (26), 5620-5626. https://doi.org/10.1039/DOTB00892C.
(45) Evtushenko, E. A.; Ryabchevskaya, E. M.; Nikitin, N. A.; Atabekov, J. G.; Karpova, O. V. Plant Virus Particles with Various Shapes as Potential Adjuvants. Sci Rep 2020, 10 (1), 10365. https://doi.org/10.1038/s41598-020-67023-4.
(46) Acosta-Ramírez, E.; Pérez-Flores, R.; Majeau, N.; Pastelin-Palacios, R.; Gil-Cruz, C.; Ramírez-Saldaña, M.; Manjarrez-Orduño, N.; Cervantes-Barragán, L.; Santos-Argumedo, L.; Flores-Romo, L.; Becker, I.; Isibasi, A.; Leclerc, D.; López-Macías, C. Translating Innate Response into Long-Lasting Antibody Response by the Intrinsic Antigen-Adjuvant Properties of Papaya Mosaic Virus. Immunology 2008, 124 (2), 186-197. https://doi.org/10.1111/j.1365-2567.2007.02753.x.
(47) Watson, D. S.; Platt, V. M.; Cao, L.; Venditto, V. J.; Szoka, F. C. Antibody Response to Polyhistidine-Tagged Peptide and Protein Antigens Attached to Liposomes via Lipid-Linked Nitrilotriacetic Acid in Mice. Clin Vaccine Immunol 2011, 18 (2), 289-297. https://doi.org/10.1128/CVI.00425-10.
(48) van Broekhoven, C. L.; Altin, J. G. The Novel Chelator Lipid 3 (Nitrilotriacetic Acid)-Ditetradecylamine (NTA (3)-DTDA) Promotes Stable Binding of His-Tagged Proteins to Liposomal Membranes: Potent Anti-Tumor Responses Induced by Simultaneously Targeting Antigen, Cytokine and Costimulatory Signals to T Cells. Biochim Biophys Acta 2005, 1716 (2), 104-116. https://doi.org/10.1016/j.bbamem.2005.09.003.
(49) Nazeri, S.; Zakeri, S.; Mehrizi, A. A.; Sardari, S.; Djadid, N. D. Measuring of IgG2c Isotype Instead of IgG2a in Immunized C57BL/6 Mice with Plasmodium Vivax TRAP as a Subunit Vaccine Candidate in Order to Correct Interpretation of Th1 versus Th2 Immune Response. Exp Parasitol 2020, 216, 107944. https://doi.org/10.1016/j.exppara.2020.107944.
(50) Schroeder, H. W.; Cavacini, L. Structure and Function of Immunoglobulins. J Allergy Clin Immunol 2010, 125 (2 0 2), S41-S52. https://doi.org/10.1016/j.jaci.2009.09.046.
(51) Tatsumi, T.; Kierstead, L. S.; Ranieri, E.; Gesualdo, L.; Schena, F. P.; Finke, J. H.; Bukowski, R. M.; Mueller-Berghaus, J.; Kirkwood, J. M.; Kwok, W. W.; Storkus, W. J. Disease-Associated Bias in T Helper Type 1 (Th1)/Th2 CD4+T Cell Responses Against MAGE-6 in HLA-DRB10401+Patients With Renal Cell Carcinoma or Melanoma. J Exp Med 2002, 196 (5), 619-628. https://doi.org/10.1084/jem.20012142.
(52) Maximiano, S.; Magalhães, P.; Guerreiro, M. P.; Morgado, M. Trastuzumab in the Treatment of Breast Cancer. BioDrugs 2016, 30 (2), 75-86. https://doi.org/10.1007/s40259-016-0162-9.
(53) Ishii, K.; Morii, N.; Yamashiro, H. Pertuzumab in the Treatment of HER2-Positive Breast Cancer: An Evidence-Based Review of Its Safety, Efficacy, and Place in Therapy. Core Evid 2019, 14, 51-70. https://doi.org/10.2147/CE.S217848.
(54) Shukla, S.; Myers, J. T.; Woods, S. E.; Gong, X.; Czapar, A. E.; Commandeur, U.; Huang, A. Y.; Levine, A. D.; Steinmetz, N. F. Plant Viral Nanoparticles-Based HER2 Vaccine: Immune Response Influenced by Differential Transport, Localization and Cellular Interactions of Particulate Carriers. Biomaterials 2017, 121, 15-27. https://doi.org/10.1016/j.biomaterials.2016.12.030.
(55) Shukla, S.; Jandzinski, M.; Wang, C.; Gong, X.; Bonk, K. W.; Keri, R. A.; Steinmetz, N. F. A Viral Nanoparticle Cancer Vaccine Delays Tumor Progression and Prolongs Survival in a HER2+Tumor Mouse Model. Advanced Therapeutics 2019, 2 (4), 1800139. https://doi.org/10.1002/adtp.201800139.
(56) Cai, H.; Shukla, S.; Wang, C.; Masarapu, H.; Steinmetz, N. F. Heterologous Prime-Boost Enhances the Antitumor Immune Response Elicited by Plant-Virus-Based Cancer Vaccine. J. Am. Chem. Soc. 2019, 141 (16), 6509-6518. https://doi.org/10.1021/jacs.9b01523.

1. A method of making a vaccine conjugate comprising conjugating a bacteriophage QBeta (“QB”) or virus nanoparticle (VNP) having one or more external lysines to a histidine-tagged antigen or antigenic peptide with a nickel nitrilotracetic acid linker (NiNTA linker), wherein the NiNTA linker binds the histidine-tagged antigen or antigenic peptide to one or more external lysines on the bacteriophage QB or VNP.

2. The method of claim 1, wherein the conjugate comprises a bacteriophage QB or Cowpea mosaic virus (CPMV).

3. (canceled)

4. The method of claim 1, wherein the VNP is or is derived from a plant virus, optionally wherein the plant virus is or is derived from a genus selected from Bromovirus, Comovirus, Tymovirus, or Sobemovirus, optionally wherein the plant virus is or is derived from Cowpea chlorotic mottle virus (CCMV), Cowpea mosaic virus (CPMV), Physalis mottle virus (PhMV), or Sesbania mosaic virus (SeMV).

5-6. (canceled)

7. The method of claim 1, wherein the antigen or antigenic peptide comprises a viral, bacterial, or tumor antigen or antigenic peptide, optionally wherein the antigenic peptide or antigen are selected from a peptide epitope from the SARS-CoV-2 S protein, or a SARS-CoV2-S receptor-binding domain (RBD-domain), a tumor antigen, an antigen to treat infectious disease, an antigen to treat cardiovascular disease, or an antigen to treat inflammation.

8. (canceled)

9. The method of claim 1, wherein a plurality of antigenic peptides or antigens are conjugated to the bacteriophage QBeta (“QB”) or the virus nanoparticle (VNP), and wherein the antigenic peptides or antigens are the same or different from each other.

10. A conjugate prepared by the method of claim 1.

11. A conjugate comprising: 1) a bacteriophage QBeta (“QB”) or virus nanoparticle (VNP) wherein the VNP is or is derived from a plant virus, having one or more external lysines; 2) a histidine-tagged antigen or antigenic peptide; and 3) a nickel nitrilotracetic acid linker (NiNTA linker), wherein the NiNTA linker binds the histidine-tagged antigen or antigenic peptide to one or more external lysines on the bacteriophage QB or VNP.

12. (canceled)

13. The conjugate of claim 11, wherein the VNP is or is derived from a plant virus, optionally wherein the plant virus is or is derived from a genus selected from Bromovirus, Comovirus, Tymovirus, or Sobemovirus, optionally wherein the plant virus is or is derived from Cowpea chlorotic mottle virus (CCMV), Cowpea mosaic virus (CPMV), Physalis mottle virus (PhMV), or Sesbania mosaic virus (SeMV).

14-15. (canceled)

16. The conjugate of claim 11, wherein the antigen or antigenic peptide comprises a viral, bacterial, or tumor antigen or antigenic peptide, optionally wherein the antigenic peptide or antigen are selected from a peptide epitope from the SARS-CoV-2 S protein, or a SARS-CoV2-S receptor-binding domain (RBD-domain).

17. (canceled)

18. The conjugate of claim 11, wherein the antigen or antigenic peptide comprises a tumor antigen, an anti-inflammatory antigen or an antigen to treat cardiovascular disease.

19. A composition comprising the conjugate of claim 11, further comprising a carrier, optionally a pharmaceutically acceptable carrier.

20. A plurality of the conjugates of claim 11.

21. The plurality of claim 20, wherein the conjugates are the same or different from each other.

22. A composition comprising the plurality of claim 20, and a carrier.

23. (canceled)

24. A method of inducing an immune response comprising administering to a subject in need thereof, an effective amount of the conjugate of claim 11.

25. A method of treating a subject in need thereof, an effective amount of the conjugate of claim 11.

26. The method of claim 25, wherein the subject is suffering from cancer and the antigen or antigenic peptide comprises a tumor antigen.

27. The method of claim 25, wherein the subject is suffering from an infectious disease, optionally SARS-CoV-2 and the antigen or antigenic peptide comprises a viral or bacterial antigen, optionally a SARS-CoV-2 S protein, or a SARS-CoV2-S receptor-binding domain (RBD-domain).

28. The method of claim 24, wherein the subject is a mammal.

29. The method of claim 27, wherein the mammal is a human subject.


SEQUENCE LISTING (9 sequences)

SEQ ID NO: 1 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 2 - protein, 374 aa, unidentified
MEQNLFALSLDDTSSVRGSLLDTKFAQTRVLLSKAMAGGDVLLDEYLYDVVNGQDFRATVAFLRTHVITGKIKVTATTNISDNSGCCLMLAINSGVRGKYSTDVYTICSQDSMTWNPGCKKNFSFTFNPNPCGDSWSAEMISRSRVRMTVICVSGWTLSPTTDVIAKLDWSIVNEKCEPTIYHLADCQNWLPLNRWMGKLTFPQGVTSEVRRMPLSIGGGAGATQAFLANMPNSWISMWRYFRGELHFEVTKMSSPYIKATVTFLIAFGNLSDAFGFYESFPHRIVQFAEVEEKCTLVFSQQEFVTAWSTQVNPRTTLEADGCPYLYAIIHDSTTGTISGDFNLGVKLVGIKDFCGIGSNPGIDGSRLLGAIAQ

SEQ ID NO: 3 - protein, 189 aa, unidentified
GPVCAEASDVYSPCMIASTPPAPFSDVTAVTFDLINGKITPVGDDNWNTHIYNPPIMNVLRTAAWKSGTIHVQLNVRGAGVKRADWDGQVFVYLRQSMNPESYDARTFVISQPGSAMLNFSFDIIGPNSGFEFAESPWANQTTWYLECVATNPRQIQQFEVNMRFDPNFRVAGNILMPPFPLSTETPPL

SEQ ID NO: 4 - protein, 188 aa, unidentified
MDSSEVVKVKQASIPAPGSILSQPNTEQSPAIVLPFQFEATTFGTAETAAQVSLQTADPITKLTAPYRHAQIVECKAILTPTDLAVSNPLTVYLAWVPANSPATPTQILRVYGGQSFVLGGAISAAKTIEVPLNLDSVNRMLKDSVTYTDTPKLLAYSRAPTNPSKIPTASIQISGRIRLSKPMLIAN

SEQ ID NO: 5 - protein, 268 aa, Sesbania mosaic virus
MAKRLSKQQLAKAIANTLETPPQPKAGRRRNRRRQRSAVQQLQPTQAGISMAPSAQGAMVRIRNPAVSSSRGGITVLTHSELSAEIGVTDSIVVSSELVMPYTVGTWLRGVAANWSKYSWLSVRYTYIPSCPSSTAGSIHMGFQYDMADTVPVSVNQLSNLRGYVSGQVWSGSAGLCFINGTRCSDTSTAISTTLDVSKLGKKWYPYKTSADYATAVGVDVNIATPLVPARLVIALLDGSSSTAVAAGRIYCTYTIQMIEPTASALNN

SEQ ID NO: 6 - protein, 6 aa, synthetic construct
HHHHHH

SEQ ID NO: 7 - protein, 8 aa, synthetic construct
HHHHHHHH

SEQ ID NO: 8 - protein, 6 aa, synthetic construct
HHHHHH

SEQ ID NO: 9 - protein, 6 aa, synthetic construct
HHHHHH